(19) United States
(12) Patent Application Publication
(10) Pub. No.: US 2025/0270493 A1
(43) Pub. Date: Aug. 28, 2025

(54) MEDIUM CULTURING AND OBSERVING DEVICE AND GAS CIRCUIT CONTROL METHOD THEREOF

(71) Applicant: HUA YUE MEDICAL TECHNOLOGY CO., LTD, Guangzhou (CN)

(72) Inventors: Zhenzhi Shi, Guangzhou (CN); Hongfeng Li, Guangzhou (CN); Chaolong Tang, Guangzhou (CN); Ting Li, Guangzhou (CN)

(21) Appl. No.: 19/066,225

(22) Filed: Feb. 28, 2025

Related U.S. Application Data
(63) Continuation-in-part of application No. PCT/CN2023/115203, filed on Aug. 28, 2023.

(30) Foreign Application Priority Data
Sep. 2, 2022 (CN) 202211076822.9

(51) Int. Cl. C12M 1/34; C12M 1/00; C12M 1/12; C12M 1/36; C12M 3/00

(52) U.S. Cl. CPC C12M 41/46; C12M 23/22; C12M 23/48; C12M 29/24; C12M 37/00; C12M 41/08; C12M 41/12; C12M 41/34; C12M 41/48

(57) ABSTRACT

Provided are medium culturing and observing device and gas circuit control method thereof. Medium culturing and observing device includes housing body, culture box body, culture turntable, heating component, rotation module, microscopic imaging module, moving module, and gas circuit system. By providing culture turntable and moving module, culture dish is driven to rotate to observation window position by rotating turntable, and imaging is performed through microscopic imaging module. Since position of culture dish may not be perfectly aligned with center of observation window, moving module can drive microscopic imaging module to move, thereby aligning microscopic imaging module with culture dish. Therefore, culture turntable only needs to rotate once to transfer culture dish that needs to be observed to observation window, thus reducing rotation frequency of culture turntable, reducing vibrations affecting medium.

### CROSS-REFERENCE TO RELATED APPLICATIONS

The present disclosure is a Continuation-In-Part Application of PCT Application No. PCT/CN2023/115203 filed on Aug. 28, 2023, which claims the priority to the Chinese patent application with the filing No. 202211076822.9 filed with the Chinese Patent Office on Sep. 2, 2022, and entitled “MEDIUM CULTURING AND OBSERVING DEVICE AND GAS CIRCUIT CONTROL METHOD THEREOF”, the contents of which are incorporated herein by reference in entirety.

### TECHNICAL FIELD

The present disclosure relates to the technical field of culture and observation, and particularly, to a medium culturing and observing device and a gas circuit control method thereof.

### BACKGROUND ART

In the field of assisted reproductive technology, it is necessary to select the most active embryos for implantation. To achieve this, in vitro culture and observation of embryos must first be performed, which is an essential task related to clinical microscopic examination and poses significant operational difficulty for operators. To achieve in vitro observation of embryos in the technology field of assisted reproductive, a culture environment capable of providing the temperature, humidity, and gas concentration required for medium culture is needed. Microscopic observation imaging of embryos is performed without disrupting the embryo culture environment, thereby avoiding unnecessary damage to embryos caused by changes in the culture environment due to microscopic observation operations to embryos.

Existing culture devices mostly adopt desktop culture devices for in vitro culture, thus enabling the medium to develop under favorable conditions. However, in the field of assisted reproductive technologies, when microscopic observation of the medium is required, the operator needs to transfer the medium from the culture device to the microscopic observation device to determine the development status of the medium. This method will lead to exposure of the medium to the external environment, where the gas environment will impact medium development.

To avoid the effects caused by exposure of the medium to the external environment, existing technology places the culturing and observing device inside a housing for culture and observation. A culture turntable is used to move culture dishes, which aligns the culture dishes with the imaging module. However, the rotation precision of the culture turntable is poor. During the rotation of the culture turntable, it is impossible to ensure that the culture dish is immediately rotated to the center of the observation window in just one turn. Multiple fine adjustments are required to align the culture dish with the imaging module. This leads to repeated vibrations of the culture dish, which in turn causes multiple vibrations of the medium, thereby affecting medium development.

In the design of integrating the culturing and observing device into one piece adopted in the prior art, the number of medium samples that can be cultured is relatively small. When the operator needs to observe more samples, expansion is not possible. Due to the single set culture mode, switching is not feasible when the operators need to perform multiple culture modes, thereby resulting in increased costs. Additionally, operating more culture devices and observing culture samples increase the required time for the operator.

### SUMMARY

To solve the above technical problems, the present disclosure provides a medium culturing and observing device that provides a stable culture environment for medium culture and facilitates precise positioning imaging during linear movement. The device includes:
a housing body, wherein an observation chamber is provided inside the housing body;
a culture box body, wherein the culture box body is mounted in the observation chamber and the culture box body is provided with an observation window;
a culture turntable, wherein the culture turntable is rotatably mounted inside the culture box body, and multiple culture dish positions corresponding to the observation window are arranged along a circumferential direction of the culture turntable;
a rotation module, wherein the rotation module is mounted in the observation chamber, a signal transmission member is mounted at a center of the rotation module, and the rotation module is connected to the culture turntable and is configured to drive the culture turntable to rotate;
multiple heating components, wherein the multiple heating components are respectively arranged in one-to-one correspondence to the observation window, the culture turntable, and the culture box body, and are configured to heat the observation window, the culture turntable, and the culture box body;
a microscopic imaging module, wherein the microscopic imaging module is provided in the observation chamber, the microscopic imaging module is configured to image a culture sample inside the culture box body through the observation window;
a moving module, wherein the moving module is mounted in the observation chamber, the microscopic imaging module is mounted on the moving module, and the moving module is configured to drive the microscopic imaging module to move linearly; and
a gas circuit system, wherein the gas circuit system is arranged in the observation chamber and is configured to supply gas to an interior of the culture box body and perform internal gas monitoring.

According to the medium culturing and observing device of the present disclosure, at least the following beneficial effects are achieved.

By providing the culture turntable and the moving module, the culture dish is driven to rotate to the observation window position by rotating the turntable, and imaging is performed through the microscopic imaging module. Since the position of the culture dish may not be perfectly aligned with the center of the observation window, the moving module can drive the microscopic imaging module to move, thereby aligning the microscopic imaging module with the culture dish. Therefore, the culture turntable only needs to rotate once to transfer the culture dish that needs to be observed to the observation window, thus reducing the rotation frequency of the culture turntable. This reduces vibrations affecting the medium and provides a stable environment for a medium culture.

According to some embodiments of the present disclosure, the culture dish position is configured to hold a culture dish. The culture dish includes a container body, wherein the container body is provided with an operation chamber having an upward-facing opening. A culture groove having an upward-facing opening is arranged inside the operation chamber. The culture groove includes a groove bottom surface and a guiding slope, wherein a lower end of the guiding slope is connected to the groove bottom surface, and an upper end of the guiding slope extends in an inclined manner towards an exterior of the culture groove. The groove bottom surface is provided with at least two culture microchambers having upward-facing openings, wherein the culture microchambers are arranged in a straight line, culture channels are provided between the culture microchambers, and the culture microchambers are in communication with the culture groove.

An injection groove and an injection channel are provided inside the operation chamber. One end of the injection channel is in communication with the culture microchamber through the culture groove. A connection port configured to connect the injection channel and the culture groove is equal to or larger than a connection port configured to connect the injection channel and the injection groove.

According to some embodiments of the present disclosure, a connection hole is provided on a bottom surface of the culture box body, wherein the rotation module is connected to the culture turntable through the connection hole. A chamber opening allowing the culture dish to pass through is provided on a top surface of the culture box body.

According to some embodiments of the present disclosure, the housing body is provided with an openable chamber door corresponding to the chamber opening.

According to some embodiments of the present disclosure, the rotation module includes a rotation motor and a rotation block. The rotation motor is mounted on a chamber wall of the observation chamber, and a rotating shaft of the rotation motor is connected to the rotation block. The rotation block is connected to a central region of the culture turntable.

According to some embodiments of the present disclosure, the microscopic imaging module includes:
a mounting bracket, wherein the mounting bracket is mounted on the moving module;
a condenser lens module, wherein the condenser lens module is mounted at one end of the mounting bracket;
a light source module, wherein the light source module is connected to the condenser lens module;
an objective lens module, wherein the objective lens module is mounted at the other end of the mounting bracket;
a tube lens module, wherein the tube lens module is mounted at the other end of the mounting bracket; and
a focusing module, wherein the focusing module is connected to the objective lens module, wherein
the condenser lens module, the observation window, the objective lens module, and the tube lens module are sequentially arranged along the same vertical line from top to bottom.

According to some embodiments of the present disclosure, the moving module includes:
a moving motor, wherein the moving motor is mounted on the chamber wall of the observation chamber;
a linear module, wherein the linear module is mounted on the chamber wall of the observation chamber, and the linear module is connected to the moving motor; and
a moving slider, wherein the moving slider is mounted on the linear module, and the microscopic imaging module is mounted on the moving slider.

According to some embodiments of the present disclosure, the gas circuit system includes a main gas intake unit, a first mixing chamber, a filter, a gas sensor unit, a gas intake diversion unit, a return gas convergence unit, a sterilization unit, a first diaphragm pump, and a first one-way valve, specifically:
the main gas intake unit includes at least two gas intake channels;
an output end of each gas intake channel is connected to the first mixing chamber;
an output end of the first mixing chamber is connected to an input end of the filter;
the first mixing chamber is provided with an accommodating cavity configured to initially mix gases with an output of each gas intake channel, and the first mixing chamber is further provided with a curved pipeline connected to the accommodating cavity for homogenizing the gas;
the filter is sequentially connected to the gas sensor unit and the gas intake diversion unit;
an input end of the culture box body is sequentially connected to a flow detection unit and a gas resistance unit, and an input end of the gas resistance unit is connected to an output end of the gas intake diversion unit;
an output end of the culture box body is connected to an input end of the return gas convergence unit;
an output end of the return gas convergence unit is connected to an input end of the sterilization unit;
the sterilization unit is connected to the first mixing chamber sequentially through the first diaphragm pump and the first one-way valve;
each gas intake channel is configured for transmitting one type of gas;
the first mixing chamber is configured for mixing the gas output from the main gas intake unit to generate a first mixed gas;
the filter is configured for filtering the first mixed gas;
the gas sensor unit is configured for detecting the gas concentration transmitted by each gas intake channel in the main gas intake unit;
the gas intake diversion unit is configured for distributing the first mixed gas; and
the sterilization unit receives the gas sent by the return gas convergence unit and performs ultraviolet sterilization.

According to some embodiments of the present disclosure, multiple culture box bodies are provided,
wherein an input end of each culture box body is sequentially connected to a flow detection unit and a gas resistance unit, and an input end of the gas resistance unit is connected to an output end of the gas intake diversion unit; and
an output end of each culture box body is connected to an input end of the return gas convergence unit.

According to some embodiments of the present disclosure, two culture box bodies are provided, namely a first box body and a second box body; and an auxiliary gas intake module is further included, specifically including the following.

The auxiliary gas intake module includes an auxiliary gas intake unit, a second mixing chamber, a second diaphragm pump, a gas concentration detection unit, and a reversing valve,
wherein the reversing valve is arranged in a connection channel between the second box body and the return gas convergence unit, a first end of the reversing valve is connected to the return gas convergence unit, and a second end of the reversing valve is connected to a first end of the second diaphragm pump;
a second end of the second diaphragm pump is connected to an input end of the second mixing chamber,
wherein the auxiliary air intake unit is provided with gas intake channels in the same number as those in the main gas intake unit, and an output end of each air intake unit is connected to an input end of the second mixing chamber; and
an output end of the second mixing chamber and the gas concentration detection unit are connected to the second box body.

According to some embodiments of the present disclosure, the gas concentration detection unit is configured for real-time detection of gas transmission data between the main gas intake unit and the culture box body;
when an abnormality occurs in the gas transmission data and a duration reaches a first preset duration, the function position of the reversing valve is switched to cut off the gas transmission between the main gas intake unit and the culture box body; and
the auxiliary air intake unit is driven to output gas to the culture box body.

According to some embodiments of the present disclosure, driving the auxiliary air intake unit to output gas to the culture box body specifically includes:
the second diaphragm pump extracting gas from the second mixing chamber and inflating the gas into the return gas convergence unit;
controlling the second diaphragm pump to enter a working state; and
extracting gas from the second mixing chamber, inflating the gas into the return gas convergence unit, wherein
the gas output from the return gas convergence unit sequentially passing through the sterilization unit, the first diaphragm pump, the first one-way valve, the first mixing chamber, the filter, the sensor unit, and the air intake diversion unit before flowing into the box body.

According to some embodiments of the present disclosure, the main gas intake unit includes at least two gas intake channels, wherein each gas intake channel includes a pressure-reducing valve, a pressure sensor, a flow sensor, a proportional valve, and a second one-way valve. Specifically,
an output end of the pressure-reducing valve is connected to the proportional valve; a pressure sensor is arranged in a connection passage between the pressure-reducing valve and the proportional valve;
an output end of the proportional valve is connected to the flow sensor;
the flow sensor is connected to the first mixing chamber via the second one-way valve; and
the opening of the proportional valve of the corresponding gas intake channel is adjusted according to the gas concentration detected by the gas sensor unit to control the output rate of each gas intake channel, so that the concentration of the first mixed gas meets the gas concentration set for the culture box body.

According to some embodiments of the present disclosure, the flow detection unit is configured for real-time detection of the flow rate of a split gas flowing into each culture box body;
the gas resistance unit is configured to adjust the gas resistance in the pipeline between the culture box body and the air intake diversion unit, wherein the gas resistance unit includes a throttle valve; and
the throttle valve is adjusted according to the flow rate of each split gas so that the flow rate of the split gas flowing into each culture box body meets a preset gas flow rate.

According to some embodiments of the present disclosure, the first diaphragm pump is configured to extract the gas sterilized by the sterilization unit and inflate the gas into the first mixing chamber; and
the gas sensor unit includes multiple gas sensors, wherein each sensor is configured to correspondingly detect the gas concentration transmitted by one gas intake channel.

According to some embodiments of the present disclosure, each sensor is configured to correspondingly detect the gas concentration transmitted by one gas intake channel and further configured to:
adjust the opening of the proportional valve of the corresponding gas intake channel according to the gas concentration transmitted by each gas intake channel so that the concentration of the first mixed gas meets the gas concentration set for the culture box body.

To solve the above problem, the present disclosure further provides a control method of a gas circuit system, including:
obtaining gas output from the main gas intake unit and mixing the gas to generate a first mixed gas, wherein the main gas intake unit includes at least two gas intake channels, and each gas intake channel is configured to transmit one type of gas;
detecting the concentration of each gas contained in the first mixed gas one by one; adjusting the output rate of the corresponding gas intake channel according to the concentration of each gas so that the concentration of the first mixed gas meets the preset gas concentration of the culture box body;
sending the first mixed gas to the culture box body;
obtaining the output gas of the culture box body, collecting the output gas, and performing sterilization treatment to generate a first circulating gas; and
mixing the first circulating gas with the gas output from the main gas intake unit and then sending it to the culture box body.

The control method of the gas circuit system of the present disclosure has at least the following beneficial effects.

By detecting the concentration of each gas contained in the first mixed gas one by one and adjusting the output rate of the corresponding gas intake channel according to the detected concentration value of each gas, the concentration of the first mixed gas flowing into the culture box body is enabled to meet the requirement for the preset gas concentration. The gas circuit control method provided by the present disclosure can achieve precise gas concentration control without the need to arrange a corresponding gas control channel for each culture box body individually. Moreover, after inputting the first mixed gas into the culture box body, the method further includes: obtaining the output gas of each culture box body, collecting the output gas, and performing sterilization treatment; and mixing the gas processed by sterilization treatment with the gas output from the main gas intake unit and sending the gas to the culture box body. By collecting and sterilizing the gas output from each culture box body before re-sending it to the culture box body, on one hand, the gas is directly sterilized, meaning that each time gas is input into the culture box body, it is effectively sterilized once, thus reducing the number of times to separately open the culture box body for sterilization and disinfection, where the safety is high. Furthermore, there is no need to provide a corresponding sterilization and disinfection module for each culture box body. On the other hand, the gas output from the culture box body can be recycled, thus ensuring proper handling of the gas output from the culture box body and improving resource utilization. This provides an optimal gas environment for medium culture.

According to some embodiments of the present disclosure, the method further includes: real-time detecting the gas transmission data between the main gas intake unit and the culture box body; and
when an abnormality in the gas transmission data occurs and the duration reaches a first preset duration, cutting off the gas transmission between the main gas intake unit and the culture unit and driving the auxiliary gas intake unit to output gas to the culture box body, wherein the auxiliary gas intake unit includes gas intake channels in the same number as those in the main gas intake unit.

According to some embodiments of the present disclosure, before sending the first mixed gas to the culture box body, the method further includes: filtering the first mixed gas.

According to some embodiments of the present disclosure, two culture box bodies are provided, and the step of sending the first mixed gas to each culture box body specifically includes:
distributing the first mixed gas to generate corresponding split gas according to the preset gas flow rate of each culture box body, and correspondingly sending it to each culture box body.

According to some embodiments of the present disclosure, the step of distributing the first mixed gas according to the preset gas flow rate of each culture box body and correspondingly sending it to each culture box body further includes:
real-time detecting the flow rate of the split gas flowing into each culture box body; and
adjusting the gas resistance of the split gas flowing into the corresponding culture box body according to the flow rate so that the flow rate of the split gas flowing into each culture box body meets the preset gas flow rate.

According to some embodiments of the present disclosure, the culture dish position is configured to hold a culture dish. The culture dish includes a container body, wherein the container body is provided with an operation chamber having an upward-facing opening. A culture groove having an upward-facing opening is arranged inside the operation chamber. The culture groove includes a groove bottom surface and a guiding slope, wherein a lower end of the guiding slope is connected to the groove bottom surface, and an upper end of the guiding slope extends in an inclined manner towards an exterior of the culture groove. The groove bottom surface is provided with at least two culture microchambers having upward-facing openings, wherein the culture microchambers are arranged in a straight line, culture channels are provided between the culture microchambers, and the culture microchambers are in communication with the culture groove.

According to some embodiments of the present disclosure, the microscopic imaging module includes:
a mounting bracket, wherein the mounting bracket is mounted on the moving module;
a condenser lens module, wherein the condenser lens module is mounted at one end of the mounting bracket;
a light source module, wherein the light source module is connected to the condenser lens module;
an objective lens module, wherein the objective lens module is mounted at the other end of the mounting bracket;
a tube lens module, wherein the tube lens module is mounted at the other end of the mounting bracket; and
a focusing module, wherein the focusing module is connected to the objective lens module, wherein
the condenser lens module, the observation window, the objective lens module, and the tube lens module are sequentially arranged along the same vertical line from top to bottom.

According to some embodiments of the present disclosure, the moving module includes:
a moving motor, wherein the moving motor is mounted on the chamber wall of the observation chamber;
a linear module, wherein the linear module is mounted on the chamber wall of the observation chamber, and the linear module is connected to the moving motor; and
a moving slider, wherein the moving slider is mounted on the linear module, and the microscopic imaging module is mounted on the moving slider.

To solve the above problem, the present disclosure further provides a terminal device, including a processor, a memory, and a computer program stored in the memory and configured to be executed by the processor, wherein the processor implements the gas circuit control method of the culture box body as described in any of the above embodiments when executing the computer program.

To solve the above problem, the present disclosure further provides a computer-readable storage medium, wherein the computer-readable storage medium includes a stored computer program, and the computer program controls the device in which the computer-readable storage medium is located to execute the gas circuit control method as described in any of the above embodiments when running.

Other features and advantages of the present disclosure will be set forth in the subsequent specification and, in part, become apparent from the specification or are understood by implementing the present disclosure.

### BRIEF DESCRIPTION OF DRAWINGS

FIG. 1 is a structural schematic diagram of a culturing and observing device provided in an embodiment of the present disclosure;

FIG. 2 is an internal structural schematic diagram of a culturing and observing device provided in an embodiment of the present disclosure;

FIG. 3 is a structural schematic diagram of a microscopic imaging module provided in an embodiment of the present disclosure;

FIG. 4 is a structural schematic diagram of a culture turntable provided in an embodiment of the present disclosure;

FIG. 5 is a rear structural schematic diagram of a culturing and observing device provided in an embodiment of the present disclosure;

FIG. 6 is a structural schematic diagram of a gas circuit system provided in an embodiment of the present disclosure;

FIG. 7 is a structural schematic diagram of a culture dish provided in an embodiment of the present disclosure;

FIG. 8 is a top view structural schematic diagram of a culture dish provided in an embodiment of the present disclosure;

FIG. 9 is a schematic diagram of an enlarged structure of FIG. 8 at position A provided in an embodiment of the present disclosure;

FIG. 10 is a structural schematic diagram of a container cover provided in an embodiment of the present disclosure;

FIG. 11 is a structural schematic diagram of a culture microchamber provided in an embodiment of the present disclosure;

FIG. 12 is a structural schematic diagram of another culture microchamber provided in an embodiment of the present disclosure;

FIG. 13 is a side view schematic diagram of a culture dish provided in an embodiment of the present disclosure; and

FIG. 14 is a flowchart of a gas circuit control method provided in an embodiment of the present disclosure.

### REFERENCE NUMERALS

1, housing body; 1a, main interaction window; 1b, instrument status detection window; 1c, instrument status indicator light; 1d, chamber door opening button; 1e, chamber door forced opening button; 1f, chamber gas concentration monitoring port; 1g, openable chamber door; 1h, first replacement chamber door; 1i, second replacement chamber door; 2, culture box body; 2a, observation window; 2b, chamber opening; 3, culture turntable; 4, rotation module; 5, microscopic imaging module; 5a, mounting bracket; 5b, condenser lens module; 5c, light source module; 5d, objective lens module; 5e, tube lens module; 5f, focusing module; 6, moving module; 66, humidification module; 8, auxiliary expansion module; 8a, gas circuit interface; 8b, communication interface; 8c, data interface;
9, culture dish; 10, container body; 11, container cover; 12, operation chamber; 13, first vertical side surface; 14, middle inclined surface; 15, second vertical side surface; 16, supporting foot; 17, limiting step; 18, handheld portion; 19, identification marking portion;
20, culture groove; 21, groove bottom surface; 22, guiding slope; 23, culture vertical surface; 24, positioning marking portion;
30, culture microchamber; 31, microchamber bottom surface; 32, operation inclined surface; 33, culture microchamber group; 34, culture channel;
40, injection groove; 41, injection channel; 42, injection limiting hole;
50, shrinkage pool;
60, gas circuit system; 61, main gas intake unit; 62, first mixing chamber; 623, filter; 64, gas sensor unit; 64a, first gas sensor; 64b, second gas sensor; 65, gas intake diversion unit; 66a, first box body; 66b, second box body; 67, return gas convergence unit; 68, sterilization unit; 69, first diaphragm pump; 610, first one-way valve; 611, pressure-reducing valve; 612, pressure sensor; 613, proportional valve; 614, flow sensor; 615, second one-way valve; 616, gas concentration detection unit; 617, auxiliary gas intake unit; 618, second mixing chamber; 619, second diaphragm pump; 620, reversing valve; 621, gas resistance unit; 622, flow detection unit.

### DETAILED DESCRIPTION OF EMBODIMENTS

The technical solutions in the embodiments of the present disclosure will be clearly and completely described below in conjunction with the drawings in the embodiments of the present disclosure. Obviously, the described embodiments are only a part of the embodiments of the present disclosure, and not all of the embodiments. Based on the embodiments in the present disclosure, all other embodiments obtained by those of ordinary skill in the art without making inventive efforts are within the scope of protection of the present disclosure.

In the description of the present disclosure, it should be understood that the terms “center”, “top”, “bottom”, “front”, “back”, “left”, “right”, “vertical”, “horizontal”, “top”, “bottom”, “inside”, “outside”, and the like indicating orientation or positional relationships are based on the orientation or positional relationships shown in the drawings. These terms are merely intended to facilitate the description of the present disclosure and simplify the description and are not intended to indicate or imply that the referenced devices or elements must have a specific orientation, be constructed in a specific orientation, or operate in a specific orientation. Therefore, these terms should not be construed as limiting the present disclosure.

In the description of the present disclosure, it is important to note that unless otherwise clearly stipulated and limited, the terms “mount”, “interconnect”, and “connect” should be understood in a broad sense, for example, it can be a fixed connection, a detachable connection, or an integral connection; it can be a mechanical connection or an electrical connection; and it can be a direct connection, an indirect connection through an intermediary, or an internal communication between two components. Those of ordinary skill in the art can understand the meanings of the above terms in the present disclosure according to specific situations.

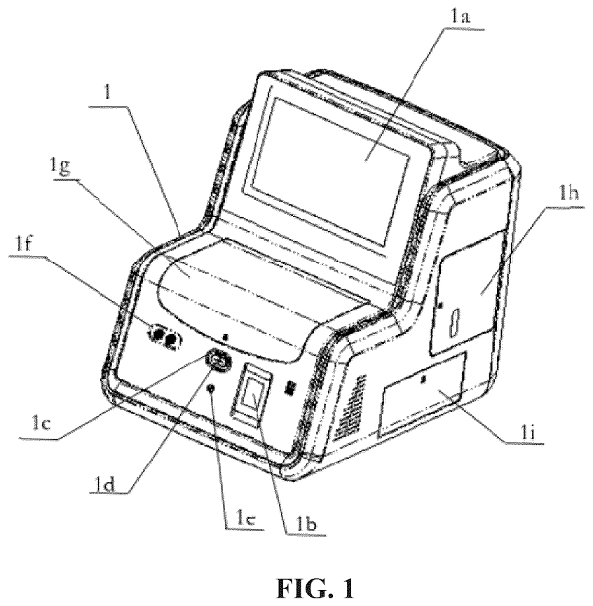

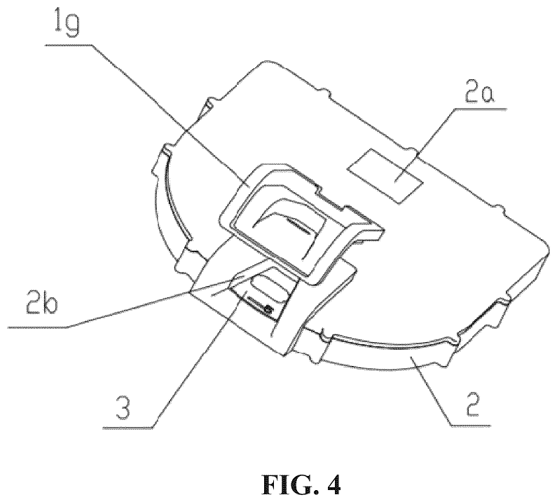

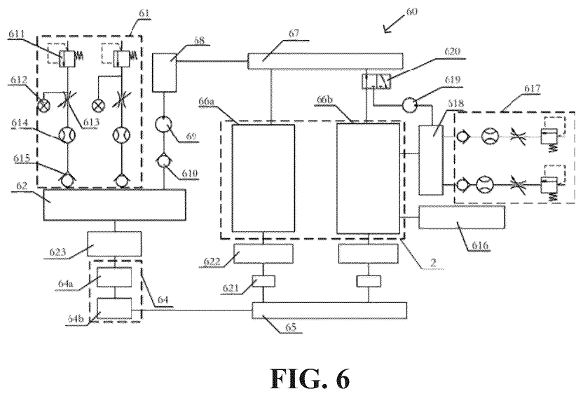

Referring to FIGS. 1 to 6, a culture medium observation device according to a preferred embodiment of the present disclosure is described. The culture medium observation device is applicable to cell culture, particularly for the culture of media. Specifically, the culture medium observation device according to the preferred embodiment of the present disclosure includes an observation structure, a culture dish, and a gas circuit system. The medium described in the present disclosure includes embryos, cells, biological particles, etc.

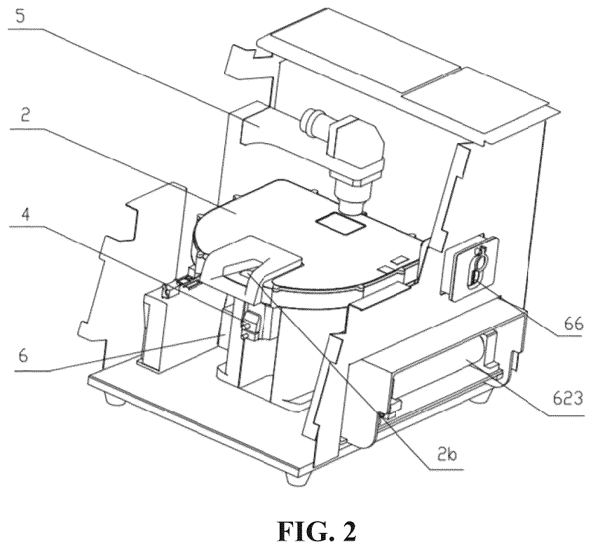

The observation structure can be specifically seen in FIG. 1 and FIG. 2. The medium culturing and observing device provided in the preferred embodiment of the present disclosure includes a housing 1, a culture box body 2, a culture turntable 3, a heating component, a rotation module 4, a microscopic imaging module 5, and a moving module 6. The housing body 1 is therein provided with an observation chamber. The culture box body 2 is mounted in the observation chamber and the culture box body 2 is provided with an observation window 2a. The culture turntable 3 can be rotatably mounted inside the culture box body 2, and multiple culture dish positions corresponding to the observation window 2a are arranged along a circumferential direction of the culture turntable 3. The rotation module 4 is mounted in the observation chamber, and a signal transmission member is mounted at a center of the rotation module, wherein the signal transmission member is configured for transmitting signals. The rotation module 4 is connected to the culture turntable 3 and is configured to drive the culture turntable 3 to rotate. The signal transmission member belongs to a contact-sliding connection application device, which can prevent problems such as wire entanglement caused by rotation and achieve accurate signal transmission between relatively rotating mechanisms. The microscopic imaging module 5 is arranged in the observation chamber, and the microscopic imaging module 5 is configured for imaging the culture samples placed in the culture dish positions inside the culture box body 2 through the observation window 2a, thereby observing the development status of the medium in the culture dish. The moving module 6 is mounted in the observation chamber, the microscopic imaging module 5 is mounted on the moving module 6, and the moving module 6 is configured to drive the microscopic imaging module 5 to move. The multiple heating components are respectively arranged in one-to-one correspondence to the observation window, the culture turntable, and the culture box body, and are configured to heat the observation window, the culture turntable, and the culture box body and to provide a favorable temperature environment for a medium culture.

It should be noted that the signal transmission member is an existing commercial signal transmission member, and details are omitted here.

It should be noted that the structure of the culture turntable is designed similarly to a “sandwich” structure, with the upper layer being the culture turntable, the middle layer being the heating component, and the lower layer being the temperature-equalizing plate. The heating component is directly attached to the culture turntable to ensure that the temperature of the culture turntable is always maintained at a constant value. The temperature-equalizing plate is directly attached to the heating device, which can reduce the gap between the heating component and the culture turntable and the temperature-equalizing plate, making the temperature more uniform and blocking the influence of the external environment temperature on the temperature of the heating component. This provides heat insulation and energy storage functions, thus reducing the temperature differences between the culture dishes. The temperature uniformity of the culture dish positions on the culture turntable is improved, thereby creating a favorable culture environment for the medium inside the culture dishes, such as embryo culture. The gap between the lower housing body of the culture box body and the culture turntable is relatively small. Heating the lower housing body of the culture box body can maintain the temperature inside the chamber at a constant value, reduce the temperature gradient difference between the culture turntable and the lower housing body of the culture box body, and reduce heat loss from the culture turntable, thus making the temperature of the culture turntable more stable and uniform. This provides a more stable environment for a medium culture.

The heating component can be arranged in the form of heating sheets or other forms, which can be arranged on multiple components of multiple modules located in the culture box body; respectively, they are located at the middle of the culture turntable assembly, the bottom surface of the lower housing body of the culture box body, and the inner surface of the chamber door of the culture box body. These correspond one-to-one. Before using the medium culturing and observing device, temperature control can be activated independently first, and then the medium culturing and observing device can be operated. The culture turntable is located between the lower housing body and the upper housing body of the culture box body. The heating component can also be heating glass. Two heating glass pieces are respectively embedded in the surface of the lower housing body and the upper housing body of the culture box body. The observation window is the region where the heating glass is embedded in the surface of the upper housing body, and the region provides an observation window for microscopic observation of the medium inside the culture dish. In addition, the module can control the heating of the heating glass according to different gas supply modes, thus preventing condensation from forming on the observation window. The inlet and outlet of the internal gas are channels for gas exchange between the culture chamber and the gas control module. The inlet and outlet of the internal gas are arranged on the lower housing body of the culture box body and can be located on the side or bottom of the lower housing body of the culture box body. This facilitates a favorable culture environment provided for the medium culturing and observing device. At the same time, it is convenient for imaging observation.

In the medium culturing and observing device provided by the preferred embodiment of the present disclosure, by providing the culture turntable 3 and the moving module 6 in the medium culturing and observing device, the culture dish is driven to rotate to the observation window position 2a by rotating the turntable, and imaging is performed through the microscopic imaging module 5. Since the position of the culture dish may not be perfectly aligned with the center of the observation window 2a, the moving module 6 can drive the microscopic imaging module 5 to move, thereby aligning the microscopic imaging module 5 with the culture dish. Therefore, the culture turntable 3 only needs to rotate once to transfer the culture dish that needs to be observed to the observation window 2a, thus reducing the rotation frequency of the culture turntable 3. This reduces vibrations affecting the medium and provides a stable culture environment for a medium culture.

In some embodiments of the present disclosure, a connection hole is provided on a bottom surface of the culture box body 2, wherein the rotation module 4 is connected to the culture turntable 3 through the connection hole. A chamber opening 2b allowing the culture dish to pass through is provided on a top surface of the culture box body 2. Specifically, the chamber opening 2b can be directly opened, and then the culture dish can be placed inside the culture box body 2.

In some embodiments of the present disclosure, the housing body 1 is provided with an openable chamber door 1g corresponding to the chamber opening 2b. The openable chamber door 1g corresponds to the chamber opening 2b, and the chamber opening 2b can be opened by directly opening the openable chamber door 1g.

In some embodiments of the present disclosure, the rotation module 4 includes a rotation motor and a rotation block. The rotation motor is mounted on a chamber wall of the observation chamber, and a rotating shaft of the rotation motor is connected to the rotation block. The rotation block is connected to a central region of the culture turntable 3.

In some embodiments of the present disclosure, the microscopic imaging module 5 includes a mounting bracket 5a, a condenser lens module 5b, a light source module 5c, an objective lens module 5d, a tube lens module 5e, and a focusing module 5f. The mounting bracket 5a is mounted on the moving module 6. The condenser lens module 5b is mounted at one end of the mounting bracket 5a. The light source module 5c is connected to the condenser lens module 5b. The objective lens module 5d is mounted at the other end of the mounting bracket 5a. The tube lens module 5e is mounted at the other end of the mounting bracket 5a. The focusing module 5f is connected to the objective lens module 5d. The condenser lens module 5b, the observation window 2a, the objective lens module 5d, and the tube lens module 5e are sequentially arranged along the same vertical line from top to bottom.

In some embodiments of the present disclosure, the moving module 6 includes a moving motor, a linear module, and a moving slider. The moving motor is mounted on the chamber wall of the observation chamber. The linear module is mounted on the chamber wall of the observation chamber, and the linear module is connected to the moving motor. The moving slider is mounted on the linear module, and the microscopic imaging module 5 is mounted on the moving slider.

It should be noted that the linear module is a linear module currently available on the market, and details are omitted here.

In some embodiments of the present disclosure, the housing body 1 is further provided with a main interaction window 1a, an instrument status detection window 1b, an instrument status indicator light 1c, a chamber door opening button 1d, a chamber door forced opening button 1e, and a chamber gas concentration monitoring port 1f.

In the present disclosure example, the housing body 1 is provided with a second replacement chamber door li corresponding to the filter 623. When the filter 623 needs to be replaced, the second replacement chamber door li can be directly opened for replacement.

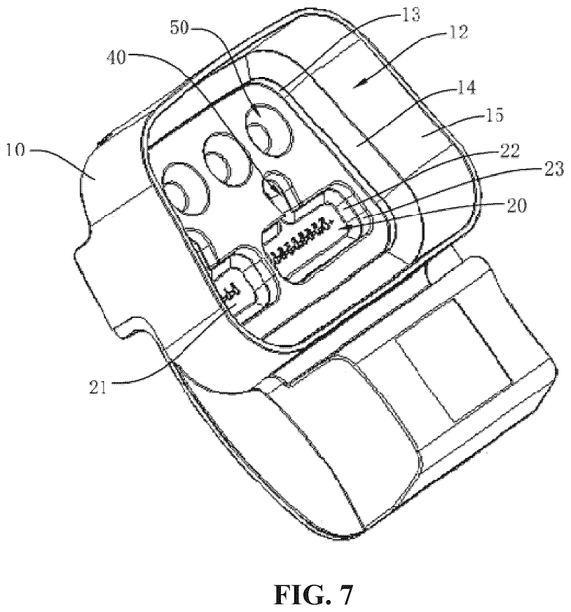

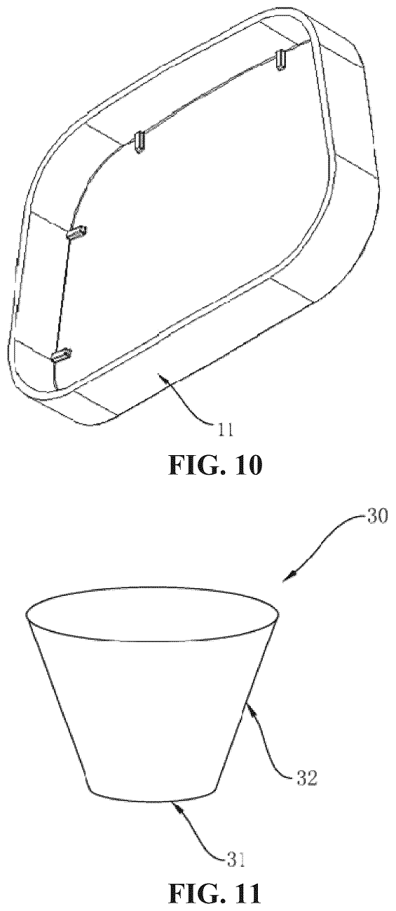

The culture dish is specifically shown in FIGS. 7 to 11. The culture dish includes a container body 10, wherein the container body 10 is provided with an operation chamber 12 having an upward-facing opening. A culture groove 20 having an upward-facing opening is arranged inside the operation chamber 12. The culture groove 20 includes a groove bottom surface 21 and a guiding slope 22, wherein a lower end of the guiding slope 22 is connected to the groove bottom surface 21, and an upper end of the guiding slope 22 extends in an inclined manner towards an exterior of the culture groove 20. The groove bottom surface 21 is provided with culture microchambers 30 having upward-facing openings, wherein culture channels 34 are provided between the culture microchambers 30, and the culture microchambers 30 are in communication with the culture groove 20.

An injection groove 40 and an injection channel 41 are provided inside the operation chamber 12. One end of the injection channel 41 is in communication with the culture microchamber 30 through the culture groove 20. A connection port configured to connect the injection channel 41 and the culture groove 20 is equal to or larger than a connection port configured to connect the injection channel 41 and the injection groove 40.

In the culture dish of the present disclosure, the container body 10 serves as a carrying container. The operation chamber 12 provides an operation space for observing, handling, and imaging the medium; and it also offers a certain level of protection for the culture microchamber 30. The culture microchamber 30 is arranged within a culture groove 20, and the culture microchamber 30 is configured to hold the culture medium. When injecting the culture fluid required for the medium into the culture groove 20, such as a culture solution, tiny bubbles can be generated during the injection process of the culture fluid. Since the culture microchamber 30 is located at the lowest position of the culture groove 20, the culture fluid first fills the culture microchamber 30. As the injection volume increases, the culture fluid overflows from the culture microchamber 30 to the groove bottom surface 21 and spreads from the groove bottom surface 21 to the lower end of the guiding slope 22. As the injection volume continues to increase, the liquid level inside the culture groove 20 rises. Since the upper end of the guiding slope 22 is inclined toward the outer side of the culture groove 20, the liquid surface formed by the culture fluid expands accordingly. During the expansion of the liquid surface, the culture fluid spreading toward the guiding slope 22 drags the tiny bubbles in the direction of the guiding slope 22. This keeps the bubbles away from the medium and ensures that there are no bubbles in the upper or edge region of the medium, thereby preventing bubbles from interfering with the culture medium. Moreover, when the microscopic imaging module observes the medium in the culture dish, it is not affected by bubbles. It is convenient for operators to observe, operate, and photograph the medium, thus providing a good culture environment for the medium culture. Additionally, it also improves the efficiency and accuracy of the observation, operation, and imaging of the medium by the operator.

It is worth noting that the microscopic imaging module, the observation window, and the medium need to be maintained on the same vertical line to ensure optimal imaging results. However, if bubbles are present in the upper or edge region of the medium in the culture dish, the microscopic imaging module is prone to interference from the bubbles during observation. This significantly affects the observation effect and efficiency, making it difficult to evaluate the developmental status of the medium and preventing timely adjustments to relevant culture factors of the medium culture, such as temperature. Therefore, the culture dish of the preferred embodiment of the present disclosure can keep bubbles away from the medium, thus ensuring optimal observation effect and efficiency.

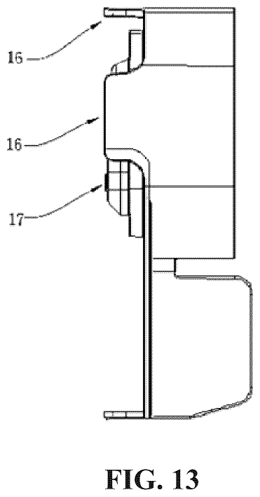

In one embodiment, as shown in FIG. 12, an operation chamber 12 is provided with a culture groove 20. The culture groove 20 is provided with at least two culture microchamber groups 33, wherein the culture microchamber groups 33 include at least two culture microchambers 30. Within the same culture microchamber group 33, adjacent culture microchambers 30 are communicated through a culture channel 34. Adjacent culture microchamber groups 33 are spaced apart.

More specifically, the operation chamber 12 is provided with a culture groove 20. The culture groove 20 is provided with two culture microchamber groups 33, wherein each culture microchamber group 33 is provided with eight culture microchambers 30.

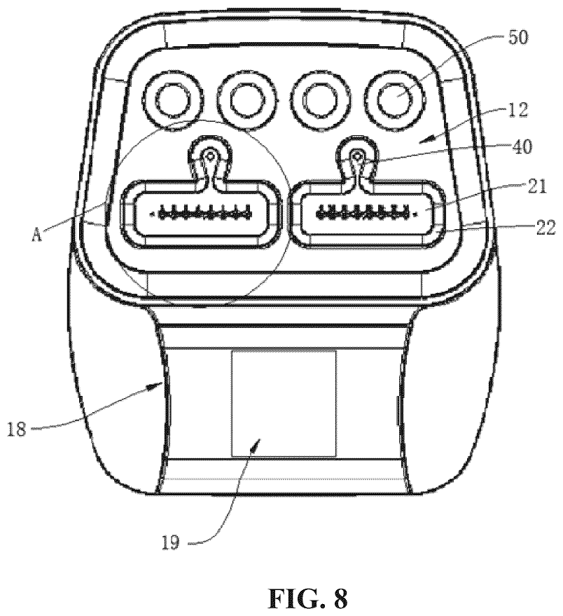

In one embodiment, as shown in FIG. 8, the operation chamber 12 is provided with at least two culture grooves 20, and adjacent culture grooves 20 are spaced apart. Each culture groove 20 is provided with one culture microchamber group 33. The culture microchamber group 33 includes at least two culture microchambers 30. Within the same culture microchamber group 33, adjacent culture microchambers 30 are communicated through a culture channel 34. The culture microchamber groups 33 are spaced apart.

More specifically, the culture microchamber group 33 is provided with eight culture microchambers 30. By providing multiple culture microchambers, the culture capacity of the culture dish is increased. Additionally, during observation, the microscopic imaging module can be fine-tuned in position through a moving module, thus reducing the need for rotation of the culture turntable. This further reduces the frequency of medium disturbances, thus ensuring the culture effect for the medium.

Further, as shown in FIGS. 7 to 9, a guiding slope 22 is arranged surrounding a groove bottom surface 21, and the outer periphery of the groove bottom surface 21 is connected to the lower end of the guiding slope 22. This improves the outward diffusion speed of the liquid surface of the culture fluid during injection, increases the traction force exerted on tiny bubbles by the expanding liquid surface, enhances the movement speed of tiny bubbles toward the guiding slope 22, and improves the effect of guiding tiny bubbles toward the guiding slope 22, thereby preventing tiny bubbles from remaining above or at the edges of the medium and causing interference. Further, this keeps the bubbles away from the medium and ensures that there are no bubbles in the upper or edge region of the medium, thereby preventing bubbles from interfering with the culture medium. Moreover, when the microscopic imaging module observes the medium in the culture dish, it is not affected by bubbles. It is convenient for operators to observe, operate, and photograph the medium. Additionally, it also improves the efficiency and accuracy of the observation, operation, and imaging of the medium by the operator.

Preferably, as shown in FIGS. 7 to 9, an angle a is formed between the guiding slope 22 and the groove bottom surface 21, where 90°<a<180°, and more preferably, 135°≤a≤165°. This provides a larger inclination for the guiding slope 22. The height of the guiding slope 22 is set between 2 mm and 6 mm to provide a greater liquid level rise space for the culture fluid, thus prolonging the processing duration of dragging tiny bubbles toward the guiding slope 22. Therefore, sufficient processing time and space are provided for the tiny bubbles to be away from the culture microchamber 30. During injection of the culture fluid, the liquid surface expands rapidly toward the guiding slope 22, which exerts a strong traction force on the tiny bubbles. This improves the effect of guiding tiny bubbles toward the guiding slope 22, thereby preventing tiny bubbles from remaining above or at the edges of the medium and causing interference. Specifically, this further reduces the impact of bubbles on observation and improves observation effect and efficiency.

Further, as shown in FIG. 7, the culture groove 20 includes a culture vertical surface 23, and the culture vertical surface 23 is arranged perpendicularly to the groove bottom surface 21. The upper end of the guiding slope 22 is connected to the lower end of the culture vertical surface 23, and the upper end of the culture vertical surface 23 is connected to the bottom surface of the operation chamber 12. The guiding slope 22 is arranged surrounding the groove bottom surface 21, and the groove bottom surface 21 is connected to the lower end of the guiding slope 22. The lower end of the culture vertical surface 23 is connected to the upper end of the guiding slope 22. Alternatively, the guiding slope 22 and the culture vertical surface 23 together form the inner side surface of the culture groove 20. The guiding slope 22 and the culture vertical surface 23 are arranged surrounding the groove bottom surface 21, respectively. The groove bottom surface 21 is connected to the lower end of the culture vertical surface 23 and the lower end of the guiding slope 22, and both side ends and the upper end of the guiding slope 22 are connected to the culture vertical surface 23. The arrangement of the culture vertical surface 23 ensures that tiny bubbles remain along the inner side wall of the culture groove 20, effectively positioning the tiny bubbles. Therefore, operators can avoid the influence of tiny bubbles when observing.

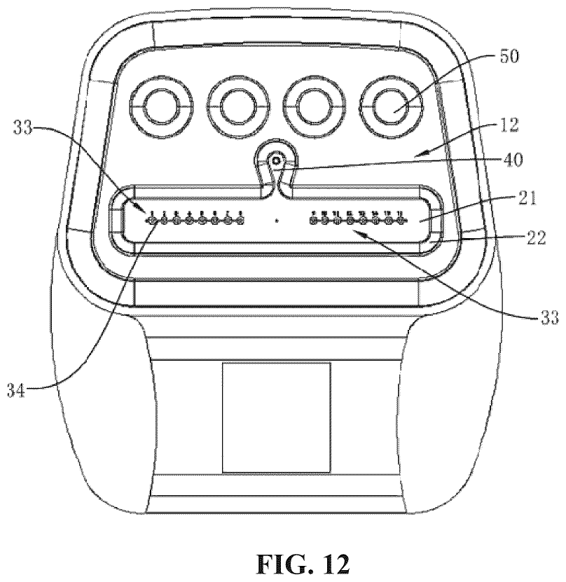

Further, as shown in FIG. 11, the inner side wall of the culture microchamber 30 includes a microchamber bottom surface 31 and an operation inclined surface 32. The upper end of the operation inclined surface 32 is inclined toward the outer circumference relative to the lower end of the operation inclined surface 32. The operation inclined surface 32 is arranged surrounding the microchamber bottom surface 31, and the lower end of the operation inclined surface 32 is connected to the microchamber bottom surface 31. An angle b is formed between the operation inclined surface 32 and the microchamber bottom surface 31, where 90°<b<180°, and more preferably, 100°≤b≤160°. The diameter of the microchamber bottom surface 31 is set between 230 μm and 300 μm. The medium diameter is approximately 160 μm to 200 μm. By increasing the bottom diameter of the culture micropore, the medium is provided with sufficient growth space without affecting imaging, thereby preventing the medium from floating during development. The inclined arrangement of the operation inclined surface 32 ensures greater stability of the culture fluid within the culture microchamber 30, which in turn stabilizes the medium during transferring the culture dish, so as to reduce the likelihood of floating.

Further, as shown in FIGS. 7 to 9, the minimum distance between the culture microchamber 30 and the inner side surface of the culture groove 20 is greater than or equal to 2 mm. On the basis of the inclined arrangement of the operation inclined surface 32 of the culture microchamber 30, the culture microchamber 30 for holding the medium is arranged away from the inner side surface of the culture groove 20. It is convenient for users to operate the culture micropore to store and take the medium, and ensures that there is enough operating space for the needle, so as to facilitate the operation from the user. This design ensures that the operating angles for all culture microchambers 30 are inclined at an angle, with a preferred operating instrument, such as an operation needle, forming an angle equal to or greater than 30 degrees relative to the vertical plane of the microchamber. At the same time, when the medium needs to inject culture fluid, tiny bubbles will be generated during the injection process of the culture fluid and flow with the culture fluid to the culture micropore region. Due to the existence of liquid surface tension, the tiny bubbles will move toward the edge region of the culture groove 20. If the distance between the inner side surface of the culture groove 20 and the culture microchamber 30 is relatively close, it will affect the imaging of the culture microchamber 30 under the microscope, thus improving the imaging and recording effect of the medium. Through angular inclination, it facilitates left-hand and right-hand operations for the operator and is also conducive to placing medium culture. This avoids the situation where the operator is restricted by the edge of the medium container during operation, which makes it difficult to inject or place the medium.

Further, as shown in FIG. 11, in the culture dish of the present disclosure, the distance from the center point of the culture microchamber 30 to the guiding slope 22 is 3 mm, the height of the guiding slope 22 is set to 3 mm, the distance from the culture groove 20 to the wall of the culture dish is 2 mm, and the distance from the center point of the culture microchamber 30 to the wall of the culture dish, i.e., the second vertical side surface 15, is 8 mm. The plane formed by the bottom surface of the culture microchamber 30 has a depth of 0.4 mm and a height of 13 mm from the plane where the upper end of the wall of the culture dish is located. Under this embodiment, through experimental verification, the present disclosure achieves the most comfortable operation angle for the operator, making the operation more convenient. For the culture microchamber 30 at both side edges, i.e., the culture microchamber 30 closest to the wall of the culture dish, the minimum operation angle formed is 60°. When operating on the culture microchamber 30 located at the edge, the operation angle gradually decreases. This solves the operational angle limitation of conventional culture dishes to the operators. The present disclosure achieves a technical implementation effect that conventional techniques cannot achieve. At the same time, in the culture dish of the present disclosure, the diameter of the microchamber bottom surface 31 is set to 260 μm, the angle b formed between the operation inclined surface 32 and the microchamber bottom surface 31 is 120°, and the depth of the culture microchamber 30 is set to 400 μm. The general diameter size of a medium during development is approximately 160-200 μm. The operation on the culture microchamber 30 forms a maximum operation angle of 90°. The cover body and the container body 10 focus on shape and configuration. The material is generally formed by polymer injection molding, such as polyester, polystyrene, PEN\PET, etc.

Further, as shown in FIG. 12, the culture groove 20 is provided with at least two culture microchamber groups 33, and the adjacent culture microchambers 30 are communicated through a culture channel 34. The adjacent culture microchambers 30 are communicated via the culture channel 34 to achieve a rapid exchange of material information between culture mediums, improve the quality of culture medium, and meet the co-culture requirements of the medium. More specifically, both end openings of the culture channel 34 are arranged on the operation inclined surface 32. The height of the culture channel 34 from the microchamber bottom surface 31 is greater than or equal to the radius of the culture medium.

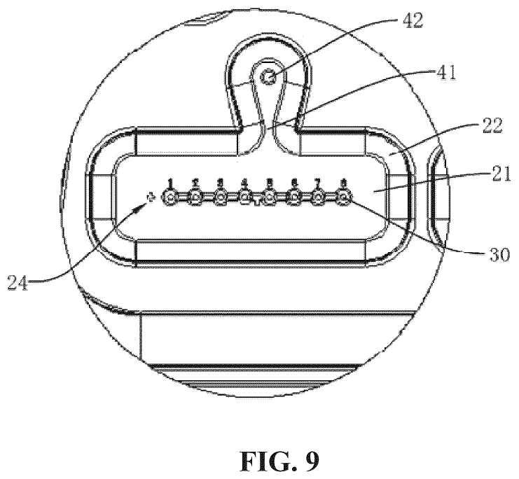

Further, multiple culture microchambers 30 are provided in the culture groove 20. As shown in FIG. 9, in one embodiment, eight culture microchambers 30 are provided, and the eight culture microchambers 30 are communicated through the culture channel 34 to achieve co-culture of multiple culture mediums. The width of the culture channel 34 is not greater than the width of the culture microchamber 30, and the depth of the culture channel 34 is smaller than the radius of the culture medium.

Further, as shown in FIGS. 7 to 9, an injection groove 40 and an injection channel 41 are provided in the operation chamber 12. One end of the injection channel 41 is communicated with the injection groove 40, and the other end of the injection channel 41 is communicated with the culture microchamber 30. The injection groove 40 and the injection channel 41 form an injection buffer zone, so that the injection needle indirectly injects into the culture groove 20, thus reducing the generation of injection bubbles. Thus, the reduction of injection bubbles avoids the impact on observation.

In one embodiment, an injection groove 40 and an injection channel 41 are provided in the operation chamber 12. One end of the injection channel 41 is communicated with the injection groove 40, and the other end of the injection channel is simultaneously communicated with multiple culture microchambers 30 through the culture groove 20.

When multiple culture microchambers 30 are provided, the injection groove 40 is simultaneously communicated with multiple injection channels 41, and the end of one injection channel 41 can be directly communicated with one or more culture microchambers 30. The culture fluid can be used to drag the tiny bubbles above or at the edge of the culture microchamber 30 at a closer distance, providing a better traction effect. Alternatively, the end of the injection channel 41 is indirectly communicated with the culture microchamber 30 through the culture groove 20.

As one of the embodiments, multiple injection grooves 40 are provided, and each injection groove 40 is respectively communicated with one or more injection channels 41 at one end. The other end of each injection channel 41 is communicated with one or more culture microchambers 30. By injecting the culture fluid, the bubbles at the upper or edge region of the culture microchamber 30 can be effectively dragged.

The injection channel 41 serves to guide the culture fluid. For example, the injection channel 41 is a pipeline, or the injection channel 41 is a groove, etc., which can achieve the guiding effect for the culture fluid.

Further, the width of the injection groove 40 is greater than the width of the injection channel 41, so that the connection between the injection groove 40 and the injection channel 41 forms a water droplet shape with the injection groove 40. When the injection needle injects liquid, the injection liquid gathers in the injection groove 40, making the connection port between the injection channel 41 and the injection groove 40 form a larger injection liquid flow rate. This enables the injection liquid flowing toward the culture groove 20 to flow into the culture microchamber 30 more evenly and provides a greater pushing force on the bubbles on the culture microchamber 30, thus preventing bubbles from staying above the culture microchamber 30.

Further, as shown in FIGS. 7 to 9, one end of the injection channel 41 is communicated with the culture microchamber 30 through the culture groove 20. The connection port between the injection channel 41 and the culture groove 20 is smaller than, equal to, or greater than the connection port between the injection channel 41 and the injection groove 40. In the embodiment, the preferred connection port between the injection channel 41 and the culture groove 20 is larger than the connection port between the injection channel 41 and the injection groove 40, so that the culture fluid can flow more evenly into the culture microchamber 30, thus reducing and avoiding the impact of bubbles on imaging of the medium inside the culture micropore. Particularly, when multiple culture microchambers 30 are arranged, the effect is more significant.

According to one embodiment, as shown in FIGS. 7 to 9, the injection channel 41 is horn-shaped, with the smaller opening end communicating with the injection groove 40 and the larger opening end communicating with the culture groove 20.

According to one embodiment, as shown in FIGS. 7 to 9, multiple culture microchambers 30 are distributed within the culture groove 20, and the injection channel 41 is connected to the middle position of the distribution region formed by the culture microchambers 30 in the longitudinal direction. The culture fluid, diffused from the injection channel 41 into each culture microchamber 30, exerts a more uniform traction force on the tiny bubbles, thus ensuring that the tiny bubbles above or at the edge regions of each culture microchamber 30 are guided toward the inner side wall of the culture microchamber 20. The injection channel 41 can also be arranged circumferentially along the culture groove 20.

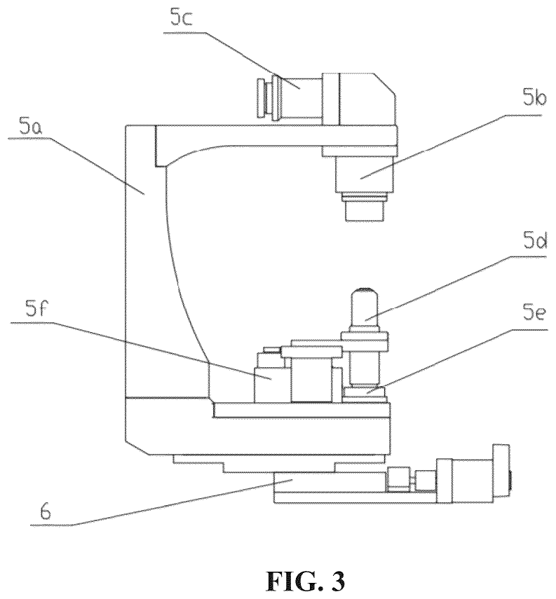

Further, as shown in FIGS. 7 to 9, an injection limiting hole 42 is provided within the injection groove 40. The injection limiting hole 42 is configured for positioning the injection pipette when injecting the culture fluid, thereby improving the stability of culture fluid injection. Further, as shown in FIG. 3, at least two culture microchambers 30 are provided within the culture groove 20, and all culture microchambers 30 form a culture medium region. A positioning marking portion 24 is provided on one side of the culture medium region. The imaging device identifies and calibrates the positioning marking portion 24 through image information, which can enable rapid positioning of each culture microchamber 30 and improve the imaging recognition efficiency of each culture microchamber 30. At least one positioning marking portion 24 is provided within each culture groove 20.

Further, the positioning marking portion 24 is arranged outside the culture groove 20, on the bottom surface of the operation chamber 12, and corresponds one-to-one with the culture microchamber 30.

Further, multiple culture grooves 20 can be provided within the operation chamber 12, and the multiple culture grooves 20 are arranged at intervals. Each culture groove 20 is provided with at least one culture microchamber 30, and the culture microchambers 30 within each culture groove 20 are arranged circumferentially around the injection limiting hole 42. The positioning marking portion corresponds one-to-one with the culture microchamber 30.

Preferably, as shown in FIGS. 7 to 9, multiple culture microchambers 30 are arranged corresponding to the positioning marking portion 24, and the positioning marking portion 24 is located at one end. By calibrating the positioning marking portion 24 at the end, the rapid positioning of each culture microchamber 30 is improved, thereby enhancing the positioning efficiency of the culture micropores.

Further, as shown in FIG. 7, a shrinkage pool 50 is provided within the operation chamber 12. The shrinkage pool 50 is configured to store the culture fluid required for the culture medium. The culture fluid can be configured to clean and remove impurities and other media around the culture medium, such as cells. The number of shrinkage pools 50 is set according to experimental requirements.

As shown in FIG. 13, the culture groove 20 and the culture microchambers 30 form a culture region. Additionally, the outer periphery of the bottom surface of the container body 10 is provided with support feet 16 for corresponding positioning and mounting with the culture turntable. The bottom surface of the container body 10 protrudes downward to form a limiting step 17 for corresponding limiting and mounting with the culture turntable. The support feet 16 and the limiting step 17 form a positioning limiting region. As shown in FIG. 8, the outer side of the container body 10 is respectively provided with a handheld portion 18 and an identification marking portion 19.

As one of the embodiments, to facilitate imaging of the medium under an observation device for evaluating the development status of the medium, the container body 10 further includes an external auxiliary region and a positioning limiting region. By adopting a segmented positioning method, which includes pre-positioning and precise positioning, the precise positioning employs a positioning separation solution in different directions. The container body is provided with positioning marking points, particularly in the culture groove where positioning marking points are arranged, thus allowing calibration and adjustment of the culture microchambers 30 under the culture medium observation device. The positioning marking points enable precise positioning of the container body 10, facilitate accurate picking and positioning of the culture container, and improve imaging quality for better evaluation of the culture medium development. The external auxiliary region includes the handheld portion 18 and the identification marking portion 19, among others. The identification marking portion 19 is affixed with relevant labels for distinguishing the container body 10 or reading related information of the container body 10. The identification marking portion 19 can be a barcode, a QR code, or a handwritten mark, allowing users or operators to intuitively identify relevant information about the container body 10. The handheld portion 18 is located on both sides of the identification region. The handheld portion 18 is designed with an arc shape to better fit the curvature of the fingers. The surface of the handheld portion 18 is treated with a matte finish to increase surface roughness. This allows for better contact feel and friction, making the gripping more secure.

The positioning and limiting region include the support feet 16 and the limiting step 17. The support feet 16 are located at the periphery of the container body 10. When container body 10 is placed into the culture turntable of the culturing and observing device, the culture turntable has a clamping position corresponding to the support feet 16, which enables pre-positioning between the support feet 16 and the culture turntable via the support feet 16, thus ensuring that the culture dish accurately enters the positioning slot. The limiting step 17 includes a limiting surface and a positioning surface. At least one side of the limiting surface is connected to one end of the positioning surface. The limiting surface is configured to restrict the position of the container body 10 in the XY plane of the culture turntable, thereby ensuring accurate positioning in the XY direction. The positioning surface is configured to restrict the position of the container body 10 in the Z-axis direction on the dedicated culture turntable, thus achieving accurate positioning in the Z-axis direction and facilitating imaging during the culture process.

The positioning marking portions 24 are in the culture groove 20, which are positioned outside the culture microchamber 30, in a cross shape, with one arranged on each side of the culture groove 20. The positioning marking portion 24 can be replaced with other shapes and can also be arranged inside the culture microchamber 30 or outside the culture groove 20. The arrangement of the positioning marking portion 24 is to pre-position, which facilitates rapid positioning by the observation device. During observation or imaging, the culture microchamber 30 is identified based on the distance between the positioning marking portion 24 and the culture microchamber 30. Then, precise positioning is achieved based on the corresponding positioning marking portion 24 of culture microchamber 30.

Further, during the injection of culture fluid, the injection needle presses against the injection limiting hole 42 inside the injection groove 40 to inject culture fluid, preventing the injection needle from moving during the injection process, thereby reducing the formation of tiny bubbles. The culture fluid first flows into the injection groove 40, and then evenly flows to the culture groove 20 through the injection channel 41 to merge with the culture fluid at the culture microchamber 30. This allows the tiny bubbles above or at the edges of the culture microchamber 30 to be pulled and diffused to the guiding slope 22. When the culture groove 20 is filled with culture fluid, the injection process for the culture groove 20 is completed.

Further, the injection limiting hole 42 is located at the center of the injection groove 40. The injection groove 40 includes an injection bottom surface and an injection side slope. The lower end of the injection side slope is connected to the injection bottom surface, and the injection side slope is arranged to surround the injection bottom surface. An angle c is formed between the injection side slope and the injection bottom surface, where 90°<c<180°. The injection limiting hole 42 is provided with an injection side wall and the injection limiting hole 42 has a height to facilitate and ensure the positioning of the injection needle.

The isolation fluid is configured to isolate the culture fluid from air, such as mineral oil, to prevent evaporation of the culture fluid.

The working process of the preferred embodiment of the culture dish in the present disclosure is as follows.

The culture fluid is injected into the culture microchamber 30, wherein during the injection process of the culture fluid, tiny bubbles are generated; as the liquid level of the culture fluid rises, the tiny bubbles in the upper region of the culture microchamber 30 are pulled to the guiding slope 22 by the culture fluid, where the liquid level of the culture fluid is higher than the bottom surface of the culture groove 20.

The culture fluid is injected into the injection groove 40 by an injection needle, wherein the injection needle presses against the injection limiting hole 42 inside the injection groove 40 to inject culture fluid, thereby preventing the injection needle from moving during the injection process, which reduces the formation of tiny bubbles. The culture fluid first flows into the injection groove 40, and then evenly flows to the culture groove 20 through the injection channel 41 to merge the culture fluid with the culture microchamber 30, which allows the tiny bubbles above the culture microchamber 30 to be pulled and diffused to the guiding slope 22; and when the culture groove 20 is filled with culture fluid, the injection process for the culture groove 20 is completed.

The isolation fluid is injected into the operation chamber 12, where the isolation fluid covers the surface of the culture fluid to form an isolation fluid layer, thereby isolating the culture fluid from air to prevent evaporation of the culture fluid;

The culture dish is equilibrated, wherein the culture dish is placed into the culture box body for equilibration treatment after placing the container cover 11 onto the opening of the container body 10. The gas circuit system is used to supply the gas to the interior to maintain the culture fluid at a stable pH and temperature, which provides a good environment for culturing the medium.

The culture dish is placed into the culture turntable of the culture box body; after equilibration, the container cover 11 and the container body 10 are removed out together; the container cover 11 is removed from the container body 10; and the medium required to culture is placed into culture microchamber 30, wherein multiple culture dishes are placed in the culture turntable in one-to-one correspondence.

After all culture dishes are placed, the chamber opening of the culture box body is closed, and the gas circuit system continues to supply gas during this period.

The medium is observed. The rotation module drives the culture turntable to rotate, which allows the culture dishes on the culture turntable to pass through the observation window on the culture box body sequentially. Since the culture microchambers are arranged in a straight line, the microscopic imaging module moves in a straight line. The operator operates the microscopic imaging module to precisely position, observe, and image each culture dish through the observation window, thereby reducing the times of the rotation of the culture dishes and providing a relatively stable culture environment for the cultured medium.

A variety of culture modes can be carried out inside the culture box body, including dry or wet culture. Further, internal gas parameters can be controlled, and for details, the following gas circuit system and control method thereof are referred to.

In some embodiments of the present disclosure, the gas circuit system 60 includes a main gas intake unit 61, a first mixing chamber 62, a filter 623, a gas sensor unit 64, a gas intake diversion unit 65, a return gas convergence unit 67, a sterilization unit 68, a first diaphragm pump 69, and a first one-way valve 610. Specifically,
the main gas intake unit 61 includes at least two gas intake channels;
an output end of each gas intake channel is connected to the first mixing chamber 62;
an output end of the first mixing chamber 62 is connected to an input end of the filter 623;
the filter 623 is sequentially connected to the gas sensor unit 64 and the gas intake diversion unit 65;
the culture box body 2 includes at least two culture box bodies, wherein an input end of each culture box body 2 is sequentially connected to a flow detection unit 622 and a gas resistance unit, an input end of the gas resistance unit is connected to an output end of the gas intake diversion unit 65;
an output end of each culture box body 2 is connected to an input end of the return gas convergence unit 67;
an output end of the return gas convergence unit 67 is connected to an input end of the sterilization unit 68;
the sterilization unit 68 is connected to the first mixing chamber 62 sequentially through the first diaphragm pump 69 and the first one-way valve 610;
each gas intake channel is configured for transmitting one type of gas;
the first mixing chamber 62 is configured for mixing the gas output from the main gas intake unit 61 to generate a first mixed gas;
the filter 623 is configured for filtering the first mixed gas;
the gas sensor unit 64 is configured for detecting the gas concentration transmitted by each gas intake channel in the main gas intake unit 61;
the gas intake diversion unit 65 is configured for distributing the first mixed gas; and
the sterilization unit 68 receives the gas sent by the return gas convergence unit 67 and performs ultraviolet sterilization.

In the embodiment of the present disclosure, the main gas intake unit 61 includes two gas intake channels, and each gas intake channel has the same structure. Taking one gas intake channel as an example, each gas inlet channel includes, from top to bottom, a pressure-reducing valve 611, a pressure sensor 612, a proportional valve 613, a flow sensor 614, and a second one-way valve 615. The output end of the second one-way valve 615 is connected to the first mixing chamber 62.

In the embodiment of the present disclosure, the provided gas circuit system can adopt a self-mixed gas supply mode. The opening of the proportional valve 613 of the corresponding gas intake channel is adjusted according to the gas concentration values detected by the gas sensor unit 64, thereby controlling the output rate of each gas intake channel. Therefore, the first mixed gas generated after the gases in each gas intake channel are mixed meets the preset gas concentration requirement of the culture box body 2. Preferably, for example, the target carbon dioxide concentration required for the gas environment of the medium culture is 6%, and the gas concentration required for oxygen is 5%. The interior of the culture box body has an atmospheric environment, and two gas sources (each with 100% concentration pure gas) are set, namely nitrogen and carbon dioxide, and one gas source is set above the inlet passage. The gas in each gas intake channel sequentially passes through the pressure-reducing valve 611, the pressure sensor 612, the flow sensor 614, the proportional valve 613, and the second one-way valve 615 before entering the first mixing chamber 62. The first mixing chamber 62 mixes the gases from each gas intake channel, so as to generate the first mixed gas and send it to the filter 623. The filter 623 filters the first mixed gas to remove impurities such as particulate matter and VOC in the first mixed gas, thereby improving the quality of the first mixed gas. Since two gas intake channels are provided in the embodiment for transmitting two gases, the gas sensor unit 64 is correspondingly provided with the gas sensors. The first gas sensor 64a is arranged as a carbon dioxide sensor, and the second gas sensor 64b is arranged as an oxygen sensor. The order of gas detection has no effect on the shape of the first mixed gas. Alternatively, the first gas sensor 64a can be arranged as an oxygen sensor, and the second gas sensor 64b can be arranged as a carbon dioxide sensor. If multiple gas types are transmitted, additional gas sensors are included accordingly. This is not limited herein. In the embodiment of the present disclosure, a carbon dioxide sensor is first used to detect the concentration of carbon dioxide in the first mixed gas. When it is determined that the concentration of carbon dioxide does not meet the preset requirement or exceeds the preset requirement value, the proportional valve 613 of the gas intake channel corresponding to the carbon dioxide is adjusted to control the output rate of the gas intake channel, thereby adjusting the concentration of carbon dioxide. After the gas concentration adjustment of the carbon dioxide is completed, an oxygen sensor obtains the concentration of oxygen in the first mixed gas. The opening size of the proportional valve 613 of the gas intake channel corresponding to the nitrogen is adjusted to achieve control of the nitrogen concentration.

As a preferred solution, the present disclosure provides a gas circuit system that can adopt a premixed gas supply mode, in which the gas concentration required by the culture box body is premixed and directly sent to the culture box body through the gas intake channel. Specifically, the gas inlet port of the gas intake channel of the nitrogen is used to introduce the premixed gas (which has been premixed to a preset concentration). Since the gas concentration introduced into the system has already been premixed, the first mixing chamber, the gas channel corresponding to the carbon dioxide, and the gas concentration sensing unit do not need to participate in the operation and are in a closed state. Further, since the gas concentration sensing unit is in a closed state, to ensure that the gas concentration introduced into the culture box body remains stable, the second diaphragm pump is also in a closed state and does not extract the culture box unit gas collected in the return gas convergence unit into the first mixing chamber. After introducing the premixed gas into the culture box body, the flow rate of the premixed gas is adjusted by regulating the opening of the proportional valve corresponding to the gas intake channel of the nitrogen. Specifically, based on the flow rate, it can be divided into a purge mode and a maintenance mode. The purge mode uses a high flow rate to ventilate the interior of the culture device, thus enabling a rapid replacement of the gas inside the culture box body. When the culture box chamber door is opened, this mode is activated. The maintenance mode uses a low flow rate to ventilate the culture box body, thus maintaining the internal gas concentration environment of the culture box body.

In the present disclosure embodiment, the first mixed gas undergoes concentration calibration by the gas sensor unit 64 and then flows into the culture box body 2 through the gas intake diversion unit 65. The gas intake diversion unit 65 has a distribution function, which is configured for distributing the first mixed gas. Specifically, an equal-flow distribution valve or a proportional-flow distribution valve can be selected based on the gas flow rate requirements of each culture box body 2.

The first mixing chamber is provided with an accommodating cavity configured to initially mix gases with an output of each gas intake channel, and the first mixing chamber is further provided with a curved pipeline connected to the accommodating cavity for homogenizing the gas.

In the embodiment of the present disclosure, the culture box body 2 includes two culture box bodies, namely the first box body 66a and the second box body 66b, wherein the second box body 66b is the aforementioned second box body. Since the gas intake flow rates of the two culture box bodies are required to be the same, a three-way distribution valve is selected in the gas intake diversion unit 65 to divide the first mixed gas into two split gases, which are respectively sent to the first box body 66a and the second box body 66b. Before entering the culture box body, the split gas also sequentially passes through a gas resistance unit 621 and a flow detection unit 622. The flow detection unit 622 is composed of a throttle valve for detecting the flow rate of the split gas flowing into the culture box body 2, thereby obtaining the gas flow flowing into the culture box body. When the gas flow flowing into the culture box body exceeds the preset gas flow rate value or is lower than the preset gas flow rate value, the throttling cross-section or throttling length of the throttle valve is adjusted to change the resistance of the pipeline between the gas intake diversion unit 65 and the corresponding culture box body, thereby adjusting the flow rate of the split gas. This further ensures that the gas flow of the split gas flowing into each culture box body remains consistent and meets the preset gas flow. In the embodiment of the present disclosure, the outputs of the first box body 66a and the second box body 66b are connected to the return gas convergence unit 67. The return gas convergence unit 67 is configured to collect the gas output from each culture box body and send it to the sterilization unit 68. The sterilization unit 68 performs ultraviolet sterilization on the gas sent from the return gas convergence unit 67. The output end of the sterilization unit 68 is connected to the first diaphragm pump 69. The first diaphragm pump 69 provides power and, when operating, extracts gas from the sterilization unit 68, where the sterilized gas in the sterilization unit 68 is extracted and inflated into the first mixing chamber 62. The operation of the first diaphragm pump 69 can produce a pressure difference between the return gas convergence unit 67 and the first mixing chamber 62, thereby forming a circulation loop.

As a preferred solution of the embodiment of the present disclosure, the right side of the second box body 66b is further connected to a gas concentration detection unit 616. The gas concentration detection unit 616 is configured to detect the concentration of each split gas delivered by the main gas intake unit 61 to the culture box body 2. When a concentration difference between the concentration of each split gas and the preset gas concentration of the culture box body 2 exceeds the preset threshold, and the concentration difference lasts for a preset duration, it is determined that there is an unrecoverable fault between the main gas intake unit 61 and the culture box body 2. The gas transmission between the main gas intake unit 61 and the culture box body 2 is then cut off. Additionally, the auxiliary gas intake unit is controlled to deliver gas to the culture box body. Specifically, the reversing valve 620 is arranged in the connection pipeline between the second box body 66b and the return gas convergence unit 67. The function position of the reversing valve 620 is driven to be switched, and the auxiliary gas intake module is connected to the circulating gas path. The auxiliary gas intake module includes, in addition to the reversing valve 620, an auxiliary gas intake unit 617, a second mixing chamber 618, a second diaphragm pump 619, and a gas concentration detection unit 616. The auxiliary gas intake unit 617 includes gas intake channels in the same number as those in the main gas intake unit 61, and the output end of each gas intake channel is connected to the second mixing chamber 618. The reversing valve 620 and the second mixing chamber 618 are connected to one end of the second diaphragm pump 619, respectively. The second mixing chamber 618 is configured to mix the gas output from each gas intake channel in the auxiliary gas intake unit 617. The second diaphragm pump 619 is configured to extract gas from the second mixing chamber 618 and inflate the gas into the return gas convergence unit 67. After the second diaphragm pump 619 inflates the gas from the second mixing chamber 618 into the return gas convergence unit 67, the first diaphragm pump extracts the gas from the return gas convergence unit 67 and inflates it into the sterilization unit 68. The gas output from the return gas convergence unit 67 sequentially passing through the sterilization unit 68, the first diaphragm pump 69, the first one-way valve 610, the first mixing chamber 62, the filter 623, the sensor unit, and the gas intake diversion unit 65 before flowing into the culture box body 2. The specific process by which the gas in the return gas convergence unit 67 passes through the above components and enters the culture box body 2 has been described in detail in the embodiments of the present disclosure and will not be repeated here.

The embodiment of the present disclosure provides a gas circuit system 60, which obtains the gas output from multiple gas intake channels and inputs it into the mixing chamber for mixing. The mixed gas is filtered, capturing and adsorbing dust particles of different particle sizes in the gas to improve the quality of the transmitted gas. By detecting the concentration of each gas contained in the first mixed gas one by one and adjusting the output rate of the corresponding gas intake channel according to the detected concentration value of each gas, the concentration of the first mixed gas flowing into the culture box body is enabled to meet the requirement for the preset gas concentration. This provides a suitable gas environment for medium culture. The gas circuit control method provided by the embodiments of the present disclosure can achieve precise gas concentration control without the need to arrange a corresponding gas control channel for each culture box body individually. In the embodiment of the present disclosure, an output end of each culture box body is connected to the return gas convergence unit 67. The method further includes collecting and sterilizing the gas output from each culture box body; and re-inputting it into the mixing chamber. On one hand, the gas is directly sterilized, meaning that each time gas is input into the culture box body. It is effectively sterilized once, thus reducing the number of times to separately open the culture box body for sterilization and disinfection, where the safety is high. Furthermore, there is no need to provide a corresponding sterilization and disinfection module for each culture box body. On the other hand, the gas output from the culture box body can be recycled, thus ensuring proper handling of the gas output from the culture box body and improving resource utilization.

Referring to FIG. 2, the medium culturing and observing device of the preferred embodiment of the present disclosure further provides a humidification module 66. The humidification module 66 includes a humidification bottle and a heating insulation module that are sequentially connected. The housing body 1 is provided with a first replacement chamber door 1h corresponding to the humidification bottle.

Specifically, when wet culture is to be implemented, there is no need to modify the internal gas circuit system. By connecting the humidification module between the intake port of the culture box body 2 and the gas circuit system, wet culture can be achieved. The humidification bottle of the humidification module can be replaced through the first replacement chamber door 1h.

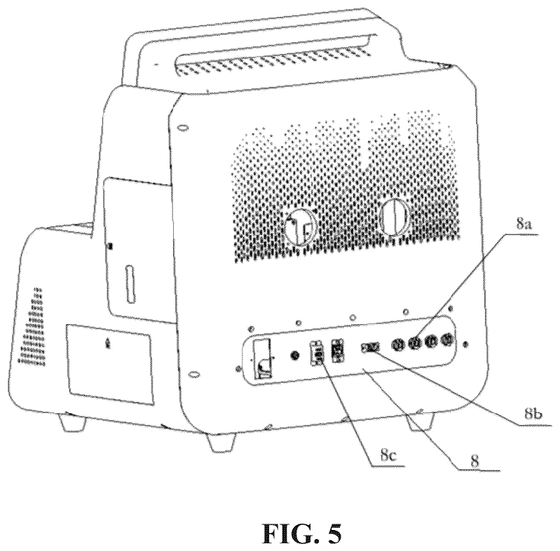

Referring to FIG. 5, the medium culturing and observing device of the preferred embodiment of the present disclosure further provides an auxiliary expansion module 8 arranged behind the medium culturing and observing device. The auxiliary expansion module 8 consists of a gas circuit interface 8a, a communication interface 8b, and a data interface 8c. The gas circuit interface 8a is located at the back, and the gas circuit interface 8a includes an intake port of the second box body and an exhaust port of the second box body, which are respectively connected to the gas circuit system through the pipeline. The pipeline is connected to the second box body, so as to supply gas to the external second box body. The communication interface 8b provides data exchange between the main module and the second box body. The data interface 8c can transmit the data information collected by the camera of the second box body to the upper computer system of the main module, thus enabling the acquisition and storage of the image data of the second box body.

It should be noted that the first mixing chamber 62 is connected to the gas circuit interface of the auxiliary expansion module and is configured to connect the external second box body 66b. The main module described above is the entire machine, which includes the first box body and the gas circuit system.

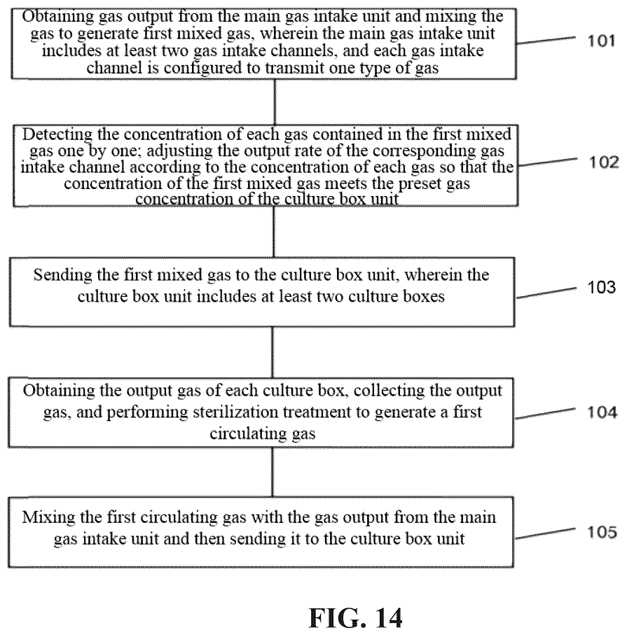

Referring to FIG. 14, the present disclosure further provides a gas circuit control method, which includes steps 101 to 105. The specific steps are as follows.
Step 101: obtaining gas output from the main gas intake unit 61 and mixing the gas to generate the first mixed gas, wherein the main gas intake unit 61 includes at least two gas intake channels, and each gas intake channel is configured to transmit one type of gas.
Step 102: detecting the concentration of each gas contained in the first mixed gas one by one; adjusting the output rate of the corresponding gas intake channel according to the concentration of each gas so that the concentration of the first mixed gas meets the preset gas concentration of the culture box body 2.
Step 103: sending the first mixed gas to the culture box body 2, wherein the culture box body 2 includes at least two culture box bodies.
Step 104: obtaining the output gas of each culture box body, collecting the output gas, and performing sterilization treatment to generate the first circulating gas.
Step 105: mixing the first circulating gas with the gas output from the main gas intake unit 61 and then sending it to the culture box body 2.

In the embodiment of the present disclosure, the gas output from the main gas intake unit 61 is mixed to generate the first mixed gas. The main gas intake unit 61 includes at least two intake paths to meet the requirements for different gas environments in the culture medium of the culture box body 2. Each gas concentration contained in the first mixed gas is detected one by one, and the output rate of the corresponding gas intake channel is adjusted according to the detected concentration value of each gas. By adjusting the output rate, the output volume of different gases in the corresponding gas intake channels changes. Therefore, the concentration of the first mixed gas, which is the mixture of the gases output from each gas intake channel, can meet the preset gas concentration requirements of the culture box body 2. Since the embodiment of the present disclosure directly detects the concentration of the first mixed gas output from each gas intake channel, the required gas environment is cultivated according to the medium. When the culture box body 2 requires multiple mixed gases and gases of different concentrations, it is only necessary to arrange the corresponding gas intake channels according to the number of gas types to generate the first mixed gas. This provides a stable gas environment for medium culture without requiring an independent gas circuit control channel arranged between each culture box body and the gas intake channel in the culture box body 2. Precise gas concentration control can also be achieved, which overcomes the problems of device redundancy, high cost, and unstable gas environment when using a single device to culture multiple different batches of culture box bodies.

In the embodiment of the present disclosure, when the first mixed gas is sent to the culture box body 2, the first mixed gas is distributed based on the gas flow requirements of each culture box body in the culture box body 2. Corresponding split gases are generated and sent to the corresponding culture box body to meet the gas flow requirements for different specifications and different culture periods. Preferably, when each split gas is sent to the corresponding culture box body, the flow rate of each split gas is also detected. The flow rate of the split gas flowing into the culture box body is obtained through the intake duration and intake flow rate. When the intake flow of the split gas is lower than the preset gas flow, the gas resistance in the pipeline between the culture box body and the split gas is correspondingly reduced to increase the flow rate of the split gas, so that the split gas flowing into the culture box body meets the preset gas flow. Correspondingly, if the intake flow of the split gas is higher than the preset gas flow, the gas resistance in the pipeline between the culture box and the split gas is increased. By reducing the resistance of the split gas, the gas flowing into the culture box body meets the preset gas flow rate.

In the embodiment of the present disclosure, after the first mixed gas is sent to the culture box body 2, the method further includes: obtaining the output gas of each culture box body, collecting the output gas, and performing sterilization treatment to generate a first circulating gas; and mixing the first circulating gas with the gas output from the main gas intake unit 61 and then sending it to the culture box body 2 for cyclic utilization. In the embodiment of the present disclosure, ultraviolet sterilization is performed on the gas output from each culture box body. On one hand, this can prevent external bacteria from entering the culture box body 2. Moreover, as the number of cycles increases, each time the gas is input into the culture box body, it can effectively serve as a sterilization and disinfection process, thus improving the safety of the gas supply and effectively reducing the number of times that the interior of the culture box body needs to be opened for sterilization and disinfection. On the other hand, the gas output from the culture box body can be recycled, thus ensuring proper handling for the gas output from the culture box body and improving resource utilization.

As a preferred solution in the embodiment of the present disclosure, real-time detection of the gas transmission data between the main gas intake unit 61 and the culture box body 2 is also included. In the embodiment of the present disclosure, the concentration of the first mixed gas transmitted between the main gas intake unit 61 and the culture box body 2 is detected in real time. When the concentration difference between the first mixed gas and the preset gas concentration of the culture box body 2 exceeds a preset threshold, it is determined that an abnormality has occurred in the gas transmission data. When the gas concentration is higher or lower than the preset gas concentration of the culture box body 2 and exceeds a preset threshold, it can be determined that the gas transmission data is abnormal. When the gas transmission data is abnormal and persists for a first preset duration, it is determined that an unrecoverable fault has occurred. The gas transmission between the main gas intake unit 61 and the culture box body 2 is immediately cut off, and the auxiliary gas intake unit 617 is driven to output gas to the culture box body 2 to provide a stable gas supply for medium culture in the culture box body. The auxiliary gas intake unit 617 includes gas intake channels in the same number as those in the main gas intake units 61 to provide the same gas supply effect as the main gas intake unit 61. When an unrecoverable fault occurs between the main gas intake unit 61 and the culture box body 2, the gas transmission between the main gas intake unit 61 and the culture box body 2 is cut off, and the auxiliary gas intake unit 617 is driven to output gas to the culture box body 2. This further ensures the reliability of the gas transmission between the main gas intake unit 61 and the culture box body 2. Using the auxiliary gas intake unit 617 as a backup intake device can also prevent drastic changes in the culture environment inside the culture box body 2 due to a failure of the main gas intake unit 61, which could affect the culture medium. This improves the risk resistance of using a single device to culture multiple different batches of culture box bodies in practical applications.

In the embodiment of the present disclosure, a gas circuit control device is further provided, including a processor, a memory, and a computer program stored in the memory and configured to be executed by the processor, wherein the processor implements the gas circuit control method described above when executing the computer program.

In the embodiment of the present disclosure, a computer-readable storage medium is further provided, wherein the computer-readable storage medium includes a stored computer program, and when the computer program is run, the device in which the computer-readable storage medium is located is controlled to execute the gas circuit control method described above.

Exemplarily, the computer program can be divided into one or more modules, wherein the one or more modules are stored in the memory and executed by the processor to complete the present disclosure. The one or more modules can be a series of computer program instruction segments capable of completing specific functions, and the instruction segments are used to describe the execution process of the computer program in the gas circuit control device.

The gas circuit control device can be computing devices such as a desktop computer, a laptop, a handheld computer, and a cloud server. The gas circuit control device can include, but is not limited to, a processor, a memory, and a display. A person skilled in the art can understand that the above components are merely exemplary of the gas circuit control device and do not constitute a limitation for the gas circuit control device. The gas circuit control device can include more or fewer components than the aforementioned components, can combine certain components, or can include different components. For example, the gas circuit control device can further include input and output devices, network access devices, buses, etc.

The processor can be a central processing unit (CPU) or other general-purpose processors, digital signal processors (DSPs), application specific integrated circuits (ASICs), field-programmable gate arrays (FPGAs), or other programmable logic devices, discrete gate or transistor logic devices, discrete hardware components, and the like. The general-purpose processor can be a microprocessor, or the processor can also be any conventional processor, and so on. The processor is the control center of the gas circuit control device and connects various parts of the gas circuit control device through various interfaces and circuits.

The memory can be configured to store the computer program and/or modules. The processor implements various functions of the gas circuit control device by running or executing computer programs and/or modules stored in the memory, and calling data stored in the memory. The memory can mainly include a program storage region and a data storage region. The program storage region can store an operating system and at least one application program required for functions (such as a sound playback function, a text conversion function, etc.). The data storage region can store data created according to the use of a mobile phone (such as audio data, text message data, etc.). Additionally, the memory can include high-speed random-access memory and non-volatile memory, such as hard disks, memory, plug-in hard disks, smart media cards (SMCs), secure digital (SD) cards, flash cards, at least one disk storage device, flash memory devices, or other volatile solid-state storage devices.

The integrated modules of the gas circuit control device can be stored on a computer-readable storage medium when implemented as software function units and sold or used as stand-alone products. Based on this understanding, all or part of the processes in the method embodiments of the present disclosure can also be implemented by instructing relevant hardware through a computer program. The computer program can be stored in a computer-readable storage medium, and when executed by a processor, the computer program can implement the steps of the various method embodiments described above. The computer program includes computer program code, and the computer program code can be in the form of source code, object code, executable files, or certain intermediate forms. The computer-readable medium can include: any entity or device capable of carrying the computer program code, recording medium, USB flash drive, mobile hard disk, magnetic disk, optical disk, computer memory, read-only memory (ROM), random access memory (RAM), electrical carrier signal, telecommunication signal and software distribution medium, etc. It should be noted that the content contained in the computer-readable medium can be appropriately increased or decreased according to the requirements of legislation and patent practice in different jurisdictions. For example, in some jurisdictions, according to legislation and patent practice, computer-readable media do not include electrical carrier signals and telecommunication signals. A person of ordinary skill in the art will be able to understand and implement them without inventive labor.

The above is merely a preferred embodiment of the present disclosure. It should be noted that for those of ordinary skill in the art, several improvements and replacements can be made without departing from the technical principles of the present disclosure, and such improvements and replacements should also be regarded as within the protection scope of the present disclosure.

1. A medium culturing and observing device, providing a stable culture environment for medium culture, comprising:
a housing body, wherein an observation chamber is provided inside the housing body;
a culture box body, wherein the culture box body is mounted in the observation chamber and the culture box body is provided with an observation window;
a culture turntable, wherein the culture turntable is rotatably mounted inside the culture box body, and multiple culture dish positions corresponding to the observation window are arranged along a circumferential direction of the culture turntable;
a rotation module, wherein the rotation module is mounted in the observation chamber, a signal transmission member is mounted at a center of the rotation module, and the rotation module is connected to the culture turntable and is configured to drive the culture turntable to rotate;
multiple heating components, wherein the multiple heating components are respectively arranged in one-to-one correspondence to the observation window, the culture turntable, and the culture box body, and are configured to heat the observation window, the culture turntable, and the culture box body;
a microscopic imaging module, wherein the microscopic imaging module is provided in the observation chamber, and the microscopic imaging module is configured to image a culture sample inside the culture box body through the observation window;
a moving module, wherein the moving module is mounted in the observation chamber, the microscopic imaging module is mounted on the moving module, and the moving module is configured to drive the microscopic imaging module to move linearly; and
a gas circuit system, wherein the gas circuit system is arranged in the observation chamber and is configured to supply gas to an interior of the culture box body and perform internal gas monitoring.

2. The medium culturing and observing device according to claim 1, wherein the culture dish position is configured to hold a culture dish; the culture dish comprises a container body, wherein the container body is provided with an operation chamber having an upward-facing opening; a culture groove having an upward-facing opening is arranged inside the operation chamber; the culture groove comprises a groove bottom surface and a guiding slope, wherein a lower end of the guiding slope is connected to the groove bottom surface, and an upper end of the guiding slope extends in an inclined manner towards an exterior of the culture groove; and the groove bottom surface is provided with at least two culture microchambers having upward-facing openings, wherein the culture microchambers are arranged in a straight line, culture channels are provided between the culture microchambers, and the culture microchambers are in communication with the culture groove.

3. The medium culturing and observing device according to claim 1, wherein the microscopic imaging module comprises:
a mounting bracket, wherein the mounting bracket is mounted on the moving module;
a condenser lens module, wherein the condenser lens module is mounted at one end of the mounting bracket;
a light source module, wherein the light source module is connected to the condenser lens module;
an objective lens module, wherein the objective lens module is mounted at another end of the mounting bracket;
a tube lens module, wherein the tube lens module is mounted at another end of the mounting bracket; and
a focusing module, wherein the focusing module is connected to the objective lens module, wherein
the condenser lens module, the observation window, the objective lens module, and the tube lens module are sequentially arranged along a same vertical line from top to bottom.

4. The medium culturing and observing device according to claim 1, wherein the moving module comprises:
a moving motor, wherein the moving motor is mounted on a chamber wall of the observation chamber;
a linear module, wherein the linear module is mounted on the chamber wall of the observation chamber, and the linear module is connected to the moving motor; and
a moving slider, wherein the moving slider is mounted on the linear module, and the microscopic imaging module is mounted on the moving slider.

5. The medium culturing and observing device according to claim 1, wherein
the gas circuit system comprises a main gas intake unit, a first mixing chamber, a filter, a gas sensor unit, a gas intake diversion unit, a return gas convergence unit, a sterilization unit, a first diaphragm pump, and a first one-way valve, wherein
the main gas intake unit comprises at least two gas intake channels;
an output end of each gas intake channel is connected to the first mixing chamber;
an output end of the first mixing chamber is connected to an input end of the filter;
the first mixing chamber is provided with an accommodating cavity configured to initially mix gases with the output end of each gas intake channel, and the first mixing chamber is further provided with a curved pipeline connected to the accommodating cavity for homogenizing the gases;
the filter is sequentially connected to the gas sensor unit and the gas intake diversion unit;
an input end of the culture box body is sequentially connected to a flow detection unit and a gas resistance unit, and an input end of the gas resistance unit is connected to an output end of the gas intake diversion unit;
an output end of the culture box body is connected to an input end of the return gas convergence unit;
an output end of the return gas convergence unit is connected to an input end of the sterilization unit;
the sterilization unit is connected to the first mixing chamber sequentially through the first diaphragm pump and the first one-way valve;
each gas intake channel is configured for transmitting one type of gas;
the first mixing chamber is configured for mixing gases output from the main gas intake unit to generate a first mixed gas;
the filter is configured for filtering the first mixed gas;
the gas sensor unit is configured for detecting a gas concentration transmitted by each gas intake channel in the main gas intake unit;
the gas intake diversion unit is configured for distributing the first mixed gas; and
the sterilization unit receives a gas sent by the return gas convergence unit and performs ultraviolet sterilization.

6. The medium culturing and observing device according to claim 5, wherein multiple culture box bodies are provided;
an input end of each culture box body is sequentially connected to the flow detection unit and the gas resistance unit, and the input end of the gas resistance unit is connected to the output end of the gas intake diversion unit; and
an output end of each culture box body is connected to the input end of the return gas convergence unit.

7. The medium culturing and observing device according to claim 6, wherein two culture box bodies are provided, comprising a first box body and a second box body; and an auxiliary gas intake module is further comprised, wherein
the auxiliary gas intake module comprises an auxiliary gas intake unit, a second mixing chamber, a second diaphragm pump, a gas concentration detection unit, and a reversing valve, wherein
the reversing valve is arranged in a connection channel between the second box body and the return gas convergence unit, a first end of the reversing valve is connected to the return gas convergence unit, and a second end of the reversing valve is connected to a first end of the second diaphragm pump; and
a second end of the second diaphragm pump is connected to an input end of the second mixing chamber, wherein
the auxiliary gas intake unit is provided with gas intake channels in the same number as those in the main gas intake unit, and an output end of each gas intake unit is connected to an input end of the second mixing chamber; and
an output end of the second mixing chamber and the gas concentration detection unit are connected to the second box body.

8. The medium culturing and observing device according to claim 5, further comprising:
the gas concentration detection unit real-time detecting gas transmission data between the main gas intake unit and the culture box body;
controlling, when an abnormality occurs in the gas transmission data and a duration reaches a first preset duration, a function position of the reversing valve to switch to cut off a gas transmission between the main gas intake unit and the culture box body; and
driving the auxiliary gas intake unit to output gas to the culture box body.

9. The medium culturing and observing device according to claim 7, wherein driving the auxiliary gas intake unit to output gas to the culture box body comprises:
the second diaphragm pump extracting gas from the second mixing chamber and inflating the gas into the return gas convergence unit;
controlling the second diaphragm pump to enter a working state; and
extracting gas from the second mixing chamber and inflating the gas into the return gas convergence unit, wherein
a gas output from the return gas convergence unit sequentially passes through the sterilization unit, the first diaphragm pump, the first one-way valve, the first mixing chamber, the filter, the sensor unit, and the gas intake diversion unit before flowing into the box body.

10. The medium culturing and observing device according to claim 7, wherein the main gas intake unit comprises at least two gas intake channels, and each gas intake channel comprises a pressure-reducing valve, a pressure sensor, a flow sensor, a proportional valve, and a second one-way valve, wherein
an output end of the pressure-reducing valve is connected to the proportional valve; the pressure sensor is arranged in a connection passage between the pressure-reducing valve and the proportional valve;
an output end of the proportional valve is connected to the flow sensor;
the flow sensor is connected to the first mixing chamber via the second one-way valve; and
an opening of the proportional valve of a corresponding gas intake channel is adjusted according to a gas concentration detected by the gas sensor unit to control an output rate of each gas intake channel, so that a concentration of the first mixed gas meets a gas concentration set for the culture box body.

11. The medium culturing and observing device according to claim 10, wherein
the flow detection unit is configured for real-time detection of a flow rate of a split gas flowing into each culture box body;
the gas resistance unit is configured to adjust a gas resistance in a pipeline between the culture box body and the gas intake diversion unit, wherein the gas resistance unit comprises a throttle valve; and
the throttle valve is adjusted according to the flow rate of each split gas so that the flow rate of the split gas flowing into each culture box body meets a preset gas flow rate.

12. A control method for the gas circuit system of the medium culturing and observing device according to claim 1,
comprising:
obtaining gas output from a main gas intake unit and mixing the gas to generate a first mixed gas, wherein the main gas intake unit comprises at least two gas intake channels, and each gas intake channel is configured to transmit one type of gas;
detecting a concentration of each gas contained in the first mixed gas one by one; adjusting an output rate of a corresponding gas intake channel according to the concentration of each gas so that a concentration of the first mixed gas meets a preset gas concentration of the culture box body;
sending the first mixed gas to the culture box body;
obtaining an output gas of the culture box body, collecting the output gas, and performing sterilization treatment to generate a first circulating gas; and
mixing the first circulating gas with a gas output from the main gas intake unit and then sending the mixed gas to the culture box body.

13. The control method for the gas circuit system according to claim 12, further comprising:
real-time detecting gas transmission data between the main gas intake unit and the culture box body; and
when an abnormality in the gas transmission data occurs and a duration reaches a first preset duration, cutting off a gas transmission between the main gas intake unit and the culture unit and driving an auxiliary gas intake unit to output gas to the culture box body, wherein the auxiliary gas intake unit comprises gas intake channels in the same number as those of the main gas intake unit.

14. The control method for the gas circuit system according to claim 12, wherein two culture box bodies are provided, and the step of sending the first mixed gas to each culture box body comprises:
distributing the first mixed gas to generate corresponding split gas according to a preset gas flow rate of each culture box body, and correspondingly sending the split gas to each culture box body.

15. The control method for the gas circuit system according to claim 14, wherein the step of distributing the first mixed gas according to a preset gas flow rate of each culture box body and correspondingly sending the split gas to each culture box body further comprises:
real-time detecting a flow rate of the split gas flowing into each culture box body; and
adjusting a gas resistance of the split gas flowing into the corresponding culture box body according to the flow rate so that the flow rate of the split gas flowing into each culture box body meets the preset gas flow rate.

16. A terminal device, comprising a processor, a memory, and a computer program stored in the memory and configured to be executed by the processor, wherein the processor implements the control method for the gas circuit system according to claim 12 when executing the computer program.

17. A computer-readable storage medium, wherein the computer-readable storage medium comprises a stored computer program, and when the computer program is run, a device in which the computer-readable storage medium is located is controlled to execute the control method for the gas circuit system according to claim 12.

18. The control method of the gas circuit system according to claim 12, wherein the culture dish position is configured to hold a culture dish; the culture dish comprises a container body, wherein the container body is provided with an operation chamber having an upward-facing opening; a culture groove having an upward-facing opening is arranged inside the operation chamber; the culture groove comprises a groove bottom surface and a guiding slope, wherein a lower end of the guiding slope is connected to the groove bottom surface, and an upper end of the guiding slope extends in an inclined manner towards an exterior of the culture groove; and the groove bottom surface is provided with at least two culture microchambers having upward-facing openings, wherein the culture microchambers are arranged in a straight line, culture channels are provided between the culture microchambers, and the culture microchambers are in communication with the culture groove.

19. The control method of the gas circuit system according to claim 12, wherein the microscopic imaging module comprises:
a mounting bracket, wherein the mounting bracket is mounted on the moving module;
a condenser lens module, wherein the condenser lens module is mounted at one end of the mounting bracket;
a light source module, wherein the light source module is connected to the condenser lens module;
an objective lens module, wherein the objective lens module is mounted at another end of the mounting bracket;
a tube lens module, wherein the tube lens module is mounted at another end of the mounting bracket; and
a focusing module, wherein the focusing module is connected to the objective lens module, wherein
the condenser lens module, the observation window, the objective lens module, and the tube lens module are sequentially arranged along a same vertical line from top to bottom.

20. The control method of the gas circuit system according to claim 12, wherein the moving module comprises:
a moving motor, wherein the moving motor is mounted on a chamber wall of the observation chamber;
a linear module, wherein the linear module is mounted on the chamber wall of the observation chamber, and the linear module is connected to the moving motor; and
a moving slider, wherein the moving slider is mounted on the linear module, and the microscopic imaging module is mounted on the moving slider.
